(19) United States
(12) Patent Application Publication
(10) Pub. No.: US 2014/0363396 A1
(43) Pub. Date: Dec. 11, 2014

(54) ONCE DAILY TREATMENT OF HEPATITIS C WITH RIBAVIRIN AND TARIBAVIRIN

(71) Applicant: KADMON PHARMACEUTICALS LLC, New York, NY (US)

(72) Inventor: Samuel Waksal, New York, NY (US)

(73) Assignee: KADMON PHARMACEUTICALS LLC, New York, NY (US)

(21) Appl. No.: 13/988,256

(22) PCT Filed: Feb. 1, 2013
(86) PCT No.: PCT/US13/24263

§ 371 Date: May 17, 2013

Related U.S. Application Data
(60) Provisional application No. 61/593,572, filed on Feb. 1, 2012.
(60) Provisional application No. 61/607,156, filed on Mar. 6, 2012.

(51) Int. Cl. A61K 31/7056; A61K 9/28; A61K 45/06

(52) U.S. Cl. CPC A61K 31/7056; A61K 45/06; A61K 9/284
USPC 424/85.7; 514/43; 514/4.3; 424/85.4

(57) ABSTRACT

Hepatitis C is treated by administering once daily ribavirin, taribavirin, other derivatives or pharmaceutically acceptable salts thereof. Hepatitis C may also be treated by administering any of the foregoing compounds once daily in combination with interferon and/or direct-acting antivirals. Once daily dosage forms administered for treating hepatitis C may comprise between 800 mg and 1400 of ribavirin. Once daily dosage forms administered for treating hepatitis C may also comprise between 800 mg and 4000 mg of taribavirin.

### FIELD OF THE INVENTION

This application relates to methods and dosage forms for the treatment of Hepatitis C with ribavirin, taribavirin and pharmaceutically acceptable salts thereof

### BACKGROUND OF THE INVENTION

Hepatitis is a medical condition defined by the inflammation of the liver. Hepatitis C, which is caused by the hepatitis C virus (HCV), afflicts more than 170 million people worldwide. No vaccine against HCV is currently available. The acute stage of HCV infection is often asymptomatic. Sometimes, however, infected individuals experience decreased appetite, fatigue, mild abdominal pain, jaundice, itching and flu-like symptoms. The great majority (up to 85%) of patients infected with HCV develop chronic hepatitis C, which means that the HCV infection persists for more than six months. Chronic hepatitis C can ultimately result in liver cirrhosis, hepatic failure or hepatocellular carcinoma (HCC), which are responsible for hundreds of thousands of deaths each year.

Ribavirin is a nucleoside analog and prodrug that is metabolized to the active 5′-phosphate in vivo. See Wu J Z et al.; Journal of Antimicrobial Chemotherapy, 2003, 52:543-546. Ribavirin has been approved for the treatment of chronic hepatitis C (CHC) virus infection in combination with pegylated interferon alfa-2a in patients 5 years of age and older with compensated liver disease and not previously treated with interferon alpha, and in CHC patients coinfected with HIV. Ribavirin has also been approved for the treatment of chronic hepatitis C (CHC) virus infection in combination with pegylated and nonpegylated interferon alfa-2b in patients 3 years of age or older with compensated liver disease. Ribavirin has also been approved for the treatment of chronic hepatitis C (CHC) virus infection in combination with consensus interferon, as further described below. Moreover, Ribavirin has been approved for the treatment of hospitalized infants and young children with severe lower tract infections due to respiratory syncytial virus.

Most commonly, hepatitis C is treated with 800 mg, 1000 mg or 1200 mg ribavirin per day. For the treatment of hepatitis C, Ribavirin is marketed in the United States as a 200 mg tablet by Roche Laboratories under the brand name Copegus®, and as a 200 mg capsule and a 40 mg/ml oral solution by Schering-Plough under the brand name Rebetol®. Generic ribavirin products are also available, including tablets containing 200, 400 and 600 mg of ribavirin and a capsule containing 200 mg of ribavirin, which are marketed by Three Rivers Pharmaceuticals, LLC under the name Ribasphere®. Three Rivers Pharmaceuticals, LLC also markets blister packs under the tradename Ribasphere® RibaPak® containing 400 mg and/or 600 mg ribavirin tablets. See U.S. Pat. No. 7,723,310. Accordingly, the daily doses of 800 mg, 1000 mg or 1200 mg ribavirin are taken in divided doses of 200, 400 and/or 600 mg, twice daily. The once-daily administration of ribavirin in previously available dosage forms in combination with interferon has also been reported. See Waizmann M and Ackermann G; Journal of Substance Abuse Treatment, 2010, 38:338-345; see also Balk et al., AASLD 2011 Annual Meeting. Thus, hepatitis C patients are often required to take a large number of medications and dosage forms every single day. The total number of capsules, tablets or other dosage forms that hepatitis C patients must take on a daily basis is often even higher because ribavirin is generally administered as a component of a multi-drug regimen (i.e., a drug cocktail). Such regimens involving the administration of multiple dosage units may decrease patient compliance. Also, ribavirin has been associated with gastrointestinal side effects, particularly where multiple pills and/or capsules are administered.

Pegylated interferon alfa-2a and nonpegylated interferon alfa-2a are marketed by Hoffman-La Roche under the names Pegasys® and Roferon®-A, respectively. Pegylated interferon alfa-2b and nonpegylated interferon alfa-2b are marketed by Schering Corporation under the names Pegintron® and Intron® A, respectively. Pegasys® is a covalent conjugate of human recombinant alfa-2a interferon with a single branched bis-monomethoxy polyethylene glycol (PEG) chain. The PEG moiety is linked at a single site to the interferon alfa moiety via a stable amide bond to lysine. Pegasys® is produced using recombinant DNA technology in which a cloned human leukocyte interferon gene is inserted into and expressed in Escherichia coli. Pegintron® is produced in a similar fashion. Roferon®-A and Intron® A are also produced in a similar fashion, except that these products are not pegylated.

In addition to interferon alfa, interferon alfacon-1 has been approved for treatment of chronic hepatitis C. At least fourteen alpha interferons (grouped into subtypes A through H) having distinct amino acid sequences have been identified. Interferon alfacon-1 is a nonnaturally-occurring, recombinant consensus polypeptide that is composed of amino acids that are common to all IFN alpha subtypes, or, at positions where there are no amino acids common to all subtypes, of amino acids which predominantly occur at these positions. See U.S. Pat. Nos. 5,372,808 and 5,541,293, which are incorporated herein by reference in their entirety. Interferon alfacon-1 is indicated for the treatment of chronic hepatitis C in patients 18 years of age or older with compensated liver disease. Use of monotherapy with interferon alfacon-1 for the treatment of hepatitis C is not recommended unless a patient is unable to take ribavirin. Combination treatment of interferon alfacon-1 with ribavirin has also been approved. Interferon alfacon-1 is marketed by Three Rivers Pharmaceuticals, LLC under the name Infergen®.

Recently, the Food and Drug Administration (FDA) also approved the HCV protease inhibitors boceprevir and telaprevir for the treatment of chronic hepatitis C genotype 1 infection, in combination with pegylated interferon alfa and ribavirin, in adult patients (18 years and older) with compensated liver disease, including cirrhosis, who are previously untreated or who have failed previous interferon and ribavirin therapy. 750 mg boceprevir and 800 mg telaprevir are administered three times a day in divided doses of 375 mg and 200 mg, respectively. Ribavirin and pegylated interferon alfa are administered at the same dosages that are used for the treatment with ribavirin and pegylated interferon alfa alone, i.e., without boceprevir or telaprevir. That means that between 400 and 600 mg of ribavirin are administered twice daily, and that pegylated interferon alfa-2a and 2b are usually administered at a dosage of 180 μg per week and about 1.5 μg/kg/week, respectively. Boceprevir is marketed by Schering Corporation under the name Victrelis™ and telaprevir is marketed by Vertex Pharmaceuticals under the name Incivek™. Boceprevir and telaprevir are often referred to as “direct-acting antivirals” (DAAs) since they directly act on, i.e., inhibit, a viral protein. Approximately 20-50% of patients with chronic hepatitis C do not respond to therapy, depending on the genotype of the HCV they are infected with.

A number of ribavirin derivatives have been developed, one of which is taribavirin. Taribavirin, also known as viramidine, is a nucleoside analogue and oral prodrug of ribavirin that is converted from taribavirin to ribavirin by adenosine deaminase. Its structural difference from ribavirin, a positively charged carboxamidine group at position 3, significantly reduces the ability of taribavirin to enter red blood cells. Because accumulation of ribavirin within red blood cells is the primary mechanism causing hemolytic anemia, taribavirin is thought to be associated with significantly less anemia. See Poordad et al., Hepatology, 2010, 52:1208-1215. Taribavirin has been developed by Valeant Pharmaceuticals International for use in combination with interferon, but it has not been approved yet for pharmaceutical use. See U.S. Pat. Nos. 7,638,496; 7,056,895; 6,930,093; 6,495,677; 6,455,508; and 6,423,695. Taribavirin is administered at a dose of 20, 25 or 30 mg/kg/day. See entry for taribavirin at http://clinicaltrials.gov/ct2; see also Poordad et al., Hepatology, 2010, 52:1208-1215. Taribavirin is also administered at a dose of 400, 600 or 800 mg twice a day. See Zeutzem, Antiviral Therapy (Meeting Report), 2005, 10:179-183. Pegylated interferon alfa is co-administered at a dose of 180 μg per week.

The present invention now provides methods of treating HCV infections by administering ribavirin, or a pharmaceutically acceptable salt thereof, once daily. In certain embodiments of the present invention, treating HCV infections includes administering ribavirin, or a pharmaceutically acceptable salt thereof, in combination with a direct-acting antiviral agent, wherein the ribavirin, or a pharmaceutically acceptable salt thereof, is administered once daily at a dose of between 800 mg and 1400 mg. In other embodiments, treating HCV infections includes administering ribavirin, or a pharmaceutically acceptable salt thereof, in combination with interferon, wherein between 800 mg and 1400 mg of ribavirin, or a pharmaceutically acceptable salt thereof, are administered once daily in a single unit. In other embodiments of the present invention, treating HCV infections includes administering ribavirin, or a pharmaceutically acceptable salt thereof, in combination with both a direct-acting antiviral agent and interferon. In other embodiments, treating HCV infections includes administering ribavirin, or a pharmaceutically acceptable salt thereof, once daily in a single unit comprising between 800 mg and 1400 mg of ribavirin, or a pharmaceutically acceptable salt thereof. The present invention also provides a pharmaceutical dosage unit comprising between 800 mg and 1400 mg of ribavirin, or a pharmaceutically acceptable salt thereof. In some embodiments of the present invention, treating HCV infections includes administering once daily dosage units that comprise both ribavirin, or a pharmaceutically acceptable salt thereof, and one or more direct-acting antivirals.

The present invention now provides methods of treating HCV infections by administering taribavirin, or a pharmaceutically acceptable salt thereof, once daily. In certain embodiments of the present invention, treating HCV infections includes administering taribavirin, or a pharmaceutically acceptable salt thereof, in combination with a direct-acting antiviral agent, wherein the taribavirin, or a pharmaceutically acceptable salt thereof, is administered once daily at a dose of between 800 mg and 1600 mg. In other embodiments, treating HCV infections includes administering taribavirin, or a pharmaceutically acceptable salt thereof, in combination with interferon, wherein between 800 mg and 1600 mg of taribavirin, or a pharmaceutically acceptable salt thereof, are administered once daily in a single unit. In other embodiments of the present invention, treating HCV infections includes administering taribavirin, or a pharmaceutically acceptable salt thereof, in combination with both a direct-acting antiviral agent and interferon. In other embodiments, treating HCV infections includes administering taribavirin, or a pharmaceutically acceptable salt thereof, once daily in a single unit comprising between 800 mg and 1600 mg of taribavirin, or a pharmaceutically acceptable salt thereof. The present invention also provides a pharmaceutical dosage unit comprising between 800 mg and 1600 mg of taribavirin, or a pharmaceutically acceptable salt thereof. In some embodiments of the present invention, treating HCV infections includes administering once daily dosage units that comprise both taribavirin, or a pharmaceutically acceptable salt thereof, and one or more direct-acting antivirals.

In a preferred embodiment, ribavirin, taribavirin or pharmaceutically acceptable salts thereof, are administered in an immediate release dosage unit. In other embodiments, ribavirin, taribavirin or pharmaceutically acceptable salts thereof, are administered in an extended release dosage unit or a combination of an immediate release and an extended release dosage unit. It is also contemplated, as described below, that other derivatives of ribavirin and taribavirin may be employed in the once-daily regimens and pharmaceutical dosage units of the present invention.

### SUMMARY OF THE INVENTION

The present invention provides a method of treating HCV infection, comprising administering ribavirin, or a pharmaceutically acceptable salt thereof, in combination with a direct-acting antiviral agent to a subject in need of such treatment, wherein the ribavirin, or a pharmaceutically acceptable salt thereof, is administered once daily at a dose of between 800 mg and 1400 mg. In some embodiments of the present invention, this dose of ribavirin, or a pharmaceutically acceptable salt thereof, is administered in one dosage unit. In other embodiments of the present invention, this dose of ribavirin, or a pharmaceutically acceptable salt thereof, is administered in two or three dosage units and each dosage unit comprises 400, 500 or 600 mg of ribavirin, or a pharmaceutically acceptable salt thereof. In other embodiments of the present invention, ribavirin, or a pharmaceutically acceptable salt thereof, is administered in the form of a tablet or a capsule. In other embodiments of the present invention, the subject is coinfected with HIV.

In certain embodiments of the present invention, the direct-acting antiviral agent is telaprevir or boceprevir. In other embodiments of the present invention, the direct-acting antiviral agent is a HCV protease inhibitor, a HCV helicase inhibitor, a HCV polymerase inhibitor, an inhibitor of HCV nonstructural proteins 4B or 5A, or an inhibitor of HCV viral ion channel forming protein p7.

The present invention further provides a method of treating HCV infection, comprising administering ribavirin, or a pharmaceutically acceptable salt thereof, in combination with interferon to a subject in need of such treatment, wherein between 800 mg and 1400 mg of ribavirin, or a pharmaceutically acceptable salt thereof, are administered once daily in a single unit. In other embodiments of the present invention, the interferon is interferon alfa-2α, interferon alfa-2β, or interferon alfacon-1. In other embodiments of the present invention, the interferon is pegylated.

The present invention also provides a method of treating HCV infection, comprising administering ribavirin, or a pharmaceutically acceptable salt thereof, in combination with both a direct-acting antiviral agent and interferon to a subject in need of such treatment, wherein the ribavirin, or a pharmaceutically acceptable salt thereof, is administered once daily at a dose of between 800 mg and 1400 mg.

The present invention further provides a method of treating HCV infection, comprising administering ribavirin, or a pharmaceutically acceptable salt thereof, to a subject in need of such treatment, wherein ribavirin, or a pharmaceutically acceptable salt thereof, is administered once daily in a single unit comprising between 800 mg and 1400 mg of ribavirin, or a pharmaceutically acceptable salt thereof. In some embodiments of the present invention, this single unit comprises 800, 1000, 1200 or 1400 mg of ribavirin, or a pharmaceutically acceptable salt thereof. In other embodiments of the present invention, this single unit is a tablet or a capsule.

The present invention further provides a pharmaceutical dosage unit comprising between 800 mg and 1400 mg of ribavirin, or a pharmaceutically acceptable salt thereof. In some embodiments of the present invention, this pharmaceutical dosage unit comprises 800, 1000, 1200 or 1400 mg of ribavirin, or a pharmaceutically acceptable salt thereof. In other embodiments of the present invention, this dosage unit is a tablet or a capsule.

The present invention provides a method of treating HCV infection, comprising administering taribavirin, or a pharmaceutically acceptable salt thereof, in combination with a direct-acting antiviral agent to a subject in need of such treatment, wherein the taribavirin, or a pharmaceutically acceptable salt thereof, is administered once daily at a dose of between 800 mg and 1600 mg. In some embodiments of the present invention, this dose of taribavirin, or a pharmaceutically acceptable salt thereof, is administered in one dosage unit. In other embodiments of the present invention, this dose of taribavirin, or a pharmaceutically acceptable salt thereof, is administered in more than one dosage units and each dosage unit comprises 200 mg of taribavirin, or a pharmaceutically acceptable salt thereof. In other embodiments of the present invention, taribavirin, or a pharmaceutically acceptable salt thereof, is administered in the form of a tablet or a capsule. In other embodiments of the present invention, the subject is coinfected with HIV.

In certain embodiments of the present invention, the direct-acting antiviral agent is telaprevir or boceprevir. In other embodiments of the present invention, the direct-acting antiviral agent is a HCV protease inhibitor, a HCV helicase inhibitor, a HCV polymerase inhibitor, an inhibitor of HCV nonstructural proteins 4B or 5A, or an inhibitor of HCV viral ion channel forming protein p7.

The present invention further provides a method of treating HCV infection, comprising administering taribavirin, or a pharmaceutically acceptable salt thereof, in combination with interferon to a subject in need of such treatment, wherein between 800 mg and 1600 mg of taribavirin, or a pharmaceutically acceptable salt thereof, are administered once daily in a single unit. In some embodiments of the present invention, the interferon is interferon alfa-2α, interferon alfa-2β, or interferon alfacon-1. In other embodiments of the present invention, the interferon is pegylated.

The present invention also provides a method of treating HCV infection, comprising administering taribavirin, or a pharmaceutically acceptable salt thereof, in combination with both a direct-acting antiviral agent and interferon to a subject in need of such treatment, wherein the taribavirin, or a pharmaceutically acceptable salt thereof, is administered once daily in one or more dosage units.

The present invention further provides a method of treating HCV infection, comprising administering taribavirin, or a pharmaceutically acceptable salt thereof, to a subject in need of such treatment, wherein taribavirin, or a pharmaceutically acceptable salt thereof, is administered once daily in a single unit comprising between 800 mg and 1600 mg of taribavirin, or a pharmaceutically acceptable salt thereof. In some embodiments of the present invention, this single unit comprises 800, 1200 or 1600 mg of ribavirin, or a pharmaceutically acceptable salt thereof. In other embodiments of the present invention, this single unit is a tablet or a capsule.

The present invention further provides a pharmaceutical dosage unit comprising between 800 mg and 1600 mg of taribavirin, or a pharmaceutically acceptable salt thereof. In some embodiments of the present invention, this pharmaceutical dosage unit comprises 800, 1200 or 1600 mg of taribavirin, or a pharmaceutically acceptable salt thereof. In other embodiments of the present invention, this dosage unit is a tablet or a capsule.

In a preferred embodiment of the present invention, ribavirin, or a pharmaceutically acceptable salt thereof, is administered in an immediate release dosage unit. In other embodiments, ribavirin, or a pharmaceutically acceptable salt thereof, is administered in an extended release dosage unit or a combination of an immediate release and an extended release dosage unit. In other embodiments, taribavirin, or a pharmaceutically acceptable salt thereof, is administered in an immediate release dosage unit, an extended release dosage unit, or a combination of an immediate release and an extended release dosage unit. It is also contemplated as described below that other derivatives of ribavirin and taribavirin may be employed in the once-daily regimens and pharmaceutical dosage units of the present invention.

It is also contemplated within the scope of the present invention that HCV infections are treated by administering once daily dosage units that comprise both ribavirin and one or more direct-acting antivirals. It is also contemplated within the scope of the present invention that HCV infections are treated by administering once daily dosage units that comprise both taribavirin and one or more direct-acting antivirals.

### DETAILED DESCRIPTION OF THE INVENTION

Current regimens and dosage forms for the treatment of hepatitis C have been associated with certain side effects. Common side effects of interferon (IFN) alfa include flu like symptoms and fatigue, a decrease in the white blood count and platelet count (a blood clotting element), depression, irritability, sleep disturbances, and anxiety as well as personality changes. The most significant side effect of ribavirin is hemolytic anemia, resulting from destruction of red blood cells. Additionally, the current ribavirin dosage regimens tend to have gastrointestinal side effects, which result in, for example, nausea, vomiting, diarrhea, abdominal pain (dyspepsia) and early satiety.

The present invention provides a method of treating HCV infection, comprising administering ribavirin in combination with a direct-acting antiviral agent to a subject in need of such treatment, wherein the ribavirin, or a pharmaceutically acceptable salt thereof, is administered once daily at a dose of between 800 mg and 1400 mg.

The present invention further provides a method of treating HCV infection, comprising administering ribavirin in combination with interferon to a subject in need of such treatment, wherein between 800 mg and 1400 mg of ribavirin, or a pharmaceutically acceptable salt thereof, are administered once daily in a single unit.

In some embodiments of the present invention, treating HCV infections includes administering ribavirin in combination with both a direct-acting antiviral agent and interferon.

The present invention further provides a method of treating HCV infection, comprising administering ribavirin, to a subject in need of such treatment, wherein ribavirin, or a pharmaceutically acceptable salt thereof, is administered once daily in a single unit comprising between 800 mg and 1400 mg of ribavirin, or a pharmaceutically acceptable salt thereof.

The present invention further provides a pharmaceutical dosage unit comprising between 800 mg and 1400 mg of ribavirin.

Ribavirin is a nucleoside analogue with antiviral activity. The chemical name of ribavirin is 1-β-D-ribofuranosyl-1H-1,2,4-triazole-3-carboxamide and ribavirin has the structural formula depicted in the formula below:

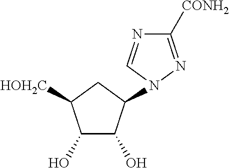 [SMILES: NC(=O)c1ncn([C@@H]2C[C@H](CO)[C@@H](O)[C@H]2O)n1]

The molecular formula of ribavirin is C₈H₁₂N₄O₅ and its molecular weight is 244.2 g/mol. Ribavirin is freely soluble in water and slightly soluble in anhydrous alcohol. See Ribasphere® Tablet Patient Prescribing Information.

In a preferred embodiment of the present invention, ribavirin is administered in an immediate release dosage unit. In other embodiments, ribavirin is administered in an extended release dosage unit or a combination of an immediate release and an extended release dosage unit.

In some embodiments of the present invention, ribavirin is administered in the form of a tablet. In other embodiments of the present invention, ribavirin is administered in the form of a capsule. The dosage forms of the present invention can be manufactured using techniques known in the art. See, e.g., U.S. Pat. Nos. 7,723,310, 7,538,094 and 6,720,000, all of which are incorporated herein by reference.

In some non-limiting embodiments of the present invention, the ribavirin dosage form is a tablet containing the following inactive ingredients: microcrystalline cellulose, lactose monohydrate, croscarmellose sodium, povidone K27-33, magnesium stearate, and purified water. The coating of the tablet contains partially hydrolyzed polyvinyl alcohol, titanium dioxide, polyethylene glycol 3350, talc, FD&C blue #2 [indigo carmine aluminum lake], and carnauba wax. Alternatively, the coating of the tablet contains partially hydrolyzed polyvinyl alcohol, titanium dioxide, polyethylene glycol 3350, talc, FD&C blue #1 [brilliant blue FCF aluminum lake], and carnauba wax. See Ribasphere® Tablet Patient Prescribing Information.

In other non-limiting embodiments of the present invention, the ribavirin dosage form consists of white pellets in a white, opaque, gelatin capsule. Each capsule contains ribavirin and the following inactive ingredients: croscarmellose sodium, NF; lactose monohydrate, NF; microcrystalline cellulose, NF; and povidone, USP. The capsule shell consists of gelatin and titanium dioxide. See Ribasphere® Capsule Patient Prescribing Information.

As a general matter, ribavirin can be administered by any convenient route commonly known in the art. Methods of administration include, but are not limited to, oral, parenteral, intradermal, intramuscular, intraperitoneal, intravenous, subcutaneous, intranasal, epidural, sublingual, intracerebral, intravaginal, transdermal, transmucosal, rectal, or topical administration, or administration by inhalation. The mode of administration is left to the discretion of the practitioner. In most instances, administration will result in the release of a compound into the bloodstream. Pulmonary administration can also be employed, e.g., by use of an inhaler or nebulizer, and formulation with an aerosolizing agent, or via perfusion in a fluorocarbon or synthetic pulmonary surfactant.

The compositions and single unit dosage forms contemplated by the present invention can therefore take the form of tablets, capsules, pills, solutions, suspensions, emulsion, powders, sustained-release formulations and the like. Because of the ease of administration, oral administration using tablets and capsules is preferred. If desired, tablets can be coated by standard aqueous or nonaqueous techniques. Such dosage forms can be prepared by any of the methods of pharmacy generally known in the art. In general, pharmaceutical compositions and dosage forms are prepared by uniformly and intimately admixing the active ingredients with liquid carriers, finely divided solid carriers, or both, and then shaping the product into the desired presentation if necessary. For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in pharmaceutical compositions provided herein is typically present in from about 50 to about 99 weight percent of the pharmaceutical composition or dosage form.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant. Disintegrants that can be used in pharmaceutical compositions and dosage forms provided herein include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB 0 SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In some embodiments of the present invention, a binder, filler or disintegrant is present in the pharmaceutical compositions provided herein from about 3 to about 60, from about 3 to about 15, from about 15 to about 25, from about 25 to about 45, or from about 45 to about 60 weight percent of the pharmaceutical composition or dosage form.

In some embodiments of the present invention, a binder is present in the pharmaceutical compositions provided herein from about 1 to about 10, from about 1 to about 3, from about 3 to about 5, from about 5 to about 8, or from about 8 to about 10 weight percent of the pharmaceutical composition or dosage form.

In some embodiments of the present invention, a disintegrant is present in the pharmaceutical compositions provided herein from about 1 to about 10, from about 1 to about 3, from about 3 to about 5, from about 5 to about 8, or from about 8 to about 10 weight percent of the pharmaceutical composition or dosage form.

In some embodiments of the present invention, a binder or disintegrant is present in the pharmaceutical compositions provided herein from about 0.1 to about 3, from about 0.1 to about 0.5, from about 0.5 to about 1, from about 1 to about 1.5, or from about 1.5 to about 3 weight percent of the pharmaceutical composition or dosage form.

In some embodiments of the present invention, a lubricant is present in the pharmaceutical compositions provided herein from about 0.1 to about 3, from about 0.1 to about 0.5, from about 0.5 to about 1, from about 1 to about 1.5, or from about 1.5 to about 3 weight percent of the pharmaceutical composition or dosage form.

In some embodiments of the present invention, microcrystalline cellulose is present in the pharmaceutical compositions provided herein from about 3 to about 60, from about 3 to about 15, from about 15 to about 25, from about 25 to about 45, or from about 45 to about 60 weight percent of the pharmaceutical composition or dosage form.

In some embodiments of the present invention, lactose monohydrate is present in the pharmaceutical compositions provided herein from about 1 to about 10, from about 1 to about 3, from about 3 to about 5, from about 5 to about 8, or from about 8 to about 10 weight percent of the pharmaceutical composition or dosage form.

In some embodiments of the present invention, crosscarmellose sodium is present in the pharmaceutical compositions provided herein from about 1 to about 10, from about 1 to about 3, from about 3 to about 5, from about 5 to about 8, or from about 8 to about 10 weight percent of the pharmaceutical composition or dosage form.

In some embodiments of the present invention, povidone K 25-33 is present in the pharmaceutical compositions provided herein from about 0.1 to about 3, from about 0.1 to about 0.5, from about 0.5 to about 1, from about 1 to about 1.5, or from about 1.5 to about 3 weight percent of the pharmaceutical composition or dosage form.

In some embodiments of the present invention, magnesium stearate is present in the pharmaceutical compositions provided herein from about 0.1 to about 3, from about 0.1 to about 0.5, from about 0.5 to about 1, from about 1 to about 1.5, or from about 1.5 to about 3 weight percent of the pharmaceutical composition or dosage form.

In some embodiments of the present invention, the dosage unit of ribavirin is an immediate release dosage unit. In other embodiments of the present invention, the dosage unit of ribavirin is an extended release dosage unit. An immediate release dosage unit is a dosage unit containing an active pharmaceutical ingredient, or drug, the release of which is not extended or controlled. Typically, these dosage units release their active pharmaceutical ingredient, or drug, relatively quickly once the dosage unit has been administered. By contrast, extended, or controlled, release dosage units release their active pharmaceutical ingredient, or drug, over an extended period of time and/or at certain location within the gastro-intestinal tract.

A compound can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with ribavirin. The present invention thus encompasses single unit dosage forms suitable for oral administration such as, but not limited to, tablets, capsules, gelcaps, and caplets that are adapted for immediate release, or that are adapted for extended and/or controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations may include extended activity of the drug, reduced dosage frequency, and increased patient compliance. In addition, controlled release formulations can be used to affect the time of onset of action or other characteristics, such as blood levels of the drug, and can thus affect the occurrence of side (e.g., adverse) effects.

Many controlled release formulations are designed to initially release an amount of drug (active ingredient) that promptly produces the desired therapeutic effect, and to gradually and continually release the remaining amount of drug to maintain this level of therapeutic effect over an extended period of time. Thus, strictly speaking, this type of formulation is a combination of an immediate release unit and an extended release unit. These and other types of combinations of an immediate release unit and an extended release unit are also within the scope of the present invention.

In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or agents.

Ribavirin tablets and capsules are currently approved for the treatment of HCV at a daily dose of between 800 mg and 1200 mg and between 800 mg and 1400 mg, respectively. The scope of the present invention covers these daily doses, but is not limited to them. Non-limiting examples of suitable daily doses, within the scope of the present invention, include any daily doses that provide a therapeutic benefit to the subject, as recognized by the person having ordinary skill in the art.

The therapeutically effective amount of ribavirin is the dose of this compound that provides a therapeutic benefit in the treatment or management of hepatitis C, delays or minimizes one or more symptoms associated with this disease, or enhances the therapeutic efficacy of another therapeutic agent used in the treatment or management of this disease. A person skilled in the art would recognize that the therapeutically effective amount may vary depending on known factors such as the pharmacodynamic and pharmacokinetic characteristics of the therapeutic compound and its mode and route of administration; the age, sex, health and weight of the subject receiving the therapeutic compound; the symptoms of the HCV infection; the frequency of the treatment and the effect desired; and the kind of the concurrent treatment. A person skilled in the art would also recognize that the therapeutically effective amount, or dose, of ribavirin can be determined based on the disclosures in this patent application and common knowledge in the art.

The amount of ribavirin that will be effective in the treatment and/or management of hepatitis C can be determined by standard clinical techniques. In vitro or in vivo assays may optionally be employed to help identify optimal dosage ranges.

A person skilled in the art may also determine the early viral response (EVR) and sustained viral response (SVR) to determine which dose of ribavirin is most appropriate in a particular case. Sustained viral response (SVR) is considered to be the defining indicator of successful treatment of a viral disease, including hepatitis C. A SVR is commonly understood to mean the absence of virus in the patient's serum six months after treatment was stopped. Early viral response (EVR) is commonly understood to mean a minimum decrease of 2 log₁₀ in the viral load (commonly determined by measuring the presence in the serum of viral DNA or RNA) during the first 12 weeks of treatment.

Suitable dosages of ribavirin for oral administration within the scope of the present invention include, but are not limited to, ribavirin doses of between 800 mg and 1400 mg administered to the subject once daily. In some embodiments of the present invention, suitable dosages include ribavirin doses of between 800 mg and 900 mg administered to the subject once daily. In other embodiments of the present invention, suitable dosages include ribavirin doses of between 900 mg and 1000 mg administered to the subject once daily. In other embodiments of the present invention, suitable dosages include ribavirin doses of between 1000 mg and 1100 mg administered to the subject once daily. In other embodiments of the present invention, suitable dosages include ribavirin doses of between 1100 mg and 1200 mg administered to the subject once daily. In other embodiments of the present invention, suitable dosages include ribavirin doses of between 1200 mg and 1400 mg administered to the subject once daily.

The present invention provides a method of treating HCV infection in a subject. The term subject, as used herein, refers to the animal being treated, wherein the animal can be a mammal such as a human.

As described below, in some embodiments of the present invention, the ribavirin dosage form is administered in combination with a direct-acting antiviral and/or interferon. Direct-acting antivirals and interferon can be manufactured and administered using techniques known to those skilled in the art. Suitable dosages and therapeutically effective amounts of direct acting antivirals and interferon can be determined as described in above in connection with the description of ribavirin.

The terms combination and co-administration and the like, as used herein, do not restrict the order in which pharmaceutical compounds are administered to a subject with a HCV infection. Ribavirin or taribavirin, or derivatives thereof, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of any of the direct-acting antivirals and interferons disclosed herein to a subject with HCV infection.

It is also contemplated within the scope of the present invention that HCV infections are treated by administering once daily dosage units that comprise both ribavirin and one or more direct-acting antivirals. Such dosage units can be manufactured and administered in ways generally known in the art. Non-limiting examples of suitable dosage forms and their routes of administration are the dosage forms and their routes of administration set forth above in connection with the description of ribavirin and below in connection with the description of direct acting antivirals. In some embodiments of the present invention, the dosage unit that comprises both ribavirin and one or more direct-acting antivirals is a tablet or a capsule.

It is contemplated within the scope of the present invention that HCV infections are treated by administering once daily a dosage unit such as a tablet that comprises ribavirin and that is scored to facilitate dividing the dosage unit. It is also contemplated within the scope of the present invention that HCV infections are treated by administering a dosage unit such as a tablet that is segmented and that contains ribavirin in one and a direct-acting antiviral agent in another segment, wherein the dosage unit is scored to facilitate dividing it into the segment containing the ribavirin and the segment containing a direct-acting antiviral agent. The present invention contemplates that in the case where such segmented dosage units are used, the ribavirin-containing segments are administered once daily. In certain embodiments of the present invention, the scored dosage unit is an immediate release dosage unit. Techniques for scoring dosage units are generally known in the art and thus not described herein in further detail.

Direct acting antivirals inhibit or prevent viral entry, viral integration into the host cell genome, viral growth and/or production by effecting the function of viral proteins. In some embodiments of the present invention, the direct-acting antiviral is boceprevir. In other embodiments of the present invention, the direct-acting antiviral is telaprevir. As set forth in more detail above and below, boceprevir and telaprevir have been approved for the treatment of chronic hepatitis C genotype 1 infection. In some embodiments of the present invention, more than one direct-acting antiviral are administered. As discussed herein, it is contemplated that these and other direct acting antivirals, as well as derivatives and pharmaceutically acceptable salts thereof, may be employed in the present invention.

HCV is highly heterogeneous. HCV is classified into eleven major genotypes (designated 1-11), many subtypes (designated a, b, c, etc.), and about 100 different strains (numbered 1,2,3, etc.) based on the genomic sequence heterogeneity. Types 1a and 1b are the most common, accounting for about 60% of global infections. They predominate in Northern Europe and North America, and in Southern and Eastern Europe and Japan, respectively. See, e.g., website of the World Health Organization at http://www.who.int/csr/disease/hepatitis/whocdsesrlyo2003/en/index2.html. Determining the genotype of the infecting RCV is important for the prediction of the response to antiviral agents because RCV genotype 1 is generally associated with a poor response to interferon alone. See Mondelli M U, Silini E.; Clinical significance of hepatitis C virus genotypes; Journal of Hepatology, 1999, 31:65-70.

HCV infection in general is commonly detected by the polymerase chain reaction (PCR), which detects the presence of HCV RNA, or by anti-HCV antibodies directed to any one of the below described proteins encoded by the HCV genome. The genotype of HCV is routinely determined by sequencing PCR-amplified regions of its genome and/or by restriction site analysis. See, e.g., Niel T. Constantine et al., Rapid Genotyping of Hepatitis C Virus; N Engl J Med, 1995, 333:800, and references cited therein. Genotyping assays are often based on the analysis of the HCV genome's 5′ untranslated region or the region encoding the nonstructural protein 5B (NSSB). See Nakatani S M et al; Comparative performance evaluation of hepatitis C virus genotyping [ . . . ]; Virology Journal, 2011, 8:459-464.

The HCV open reading frame (ORF) contains 9024 to 9111 nucleotides, depending on the specific HCV genotype. The ORF encodes a polyprotein precursor of about 3,000 amino acids. This precursor protein is cleaved by both cellular and viral proteases to 10 proteins, including 3 structural proteins (C or core, E1 and E2), a small protein, p7, whose function has not yet been definitively defined, and 6 nonstructural (NS) proteins (NS2, NS3, NS4A, NS4B, NS5A and NS5B). The NS2 protease cleaves the polyprotein precursor at the NS2/NS3 boundary. NS3 consists of an N-terminal serine protease domain and a C-terminal helicase domain. NS3 forms a non-covalent complex with the NS4A, and cleaves the polyprotein precursor at four locations: NS3/4A (self cleavage), NS4A/4B, NS4B/5A, and NS5A/5B. See, e.g., Tan S L (editor); Hepatitis C Viruses: Genomes and Molecular Biology; Horizon Bioscience, Norfolk (UK) (2006). The NS3/4A serine protease also contributes to the ability of HCV to evade early innate immune responses. NS3/4A has been shown to block virus induced activation of IFN regulatory factor 3 (IRF-3), a transcription factor playing a critical role in the induction of type-1 IFNs.

NS4B is a 27-kDa membrane protein that is primarily involved in the formation of membrane vesicles—also named membranous web—which is used as scaffold for the assembly of the HCV replication complex. In addition, NS4B contains NTPase and RNA binding activities, as well as anti-apoptotic properties.

Non-limiting examples of the types of direct acting antivirals within the scope of the present invention include inhibitors of the HCV proteases NS2 and NS3/4A, inhibitors of the HCV helicase NS3, inhibitors of the nonstructural HCV proteins NS4B and NS5A, inhibitors of the HCV polymerase NS5B and inhibitors of the viral ion channel forming protein p7. Direct acting antivirals within the scope of the present invention also include IRES (internal ribosomal entry site) inhibitors and HCV entry inhibitors. Non-limiting representative examples of each of these types of direct-acting antivirals, all of which are within the scope of the present invention, are provided below.

Inhibitors of the HCV NS3/4A protease include, without limitation, boceprevir and telaprevir (VX-950). The chemical name of boceprevir is (1R,5S)—N-[3-Amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide. The molecular formula of boceprevir is C₂₇H₄₅N₅O₅ and its molecular weight is 519.7 g/mol. Boceprevir has the following structural formula:

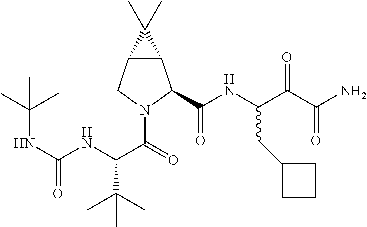 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

Boceprevir is manufactured as an approximately equal mixture of two diastereomers. Boceprevir is a white to off-white amorphous powder. It is freely soluble in methanol, ethanol and isopropanol and slightly soluble in water. See Victrelis™ Capsule Patient Prescribing Information.

The chemical name of telaprevir (VX-950) is (1S,3aR,6aS)-2-[(2S)-2-({(2 S)-2-cyclohexyl-2-[(pyrazin-2-ylcarbonyl)amino]acetyl}amino)-3,3-dimethylbutanoyl]-N-[(3S)-1-(cyclopropylamino)-1,2-dioxohexan-3-yl]-3,3a,4,5,6,6a-hexahydro-1H-cyclopenta[c]pyrrole-1-carboxamide. Its molecular formula is C₃₆H₅₃N₇O₆ and its molecular weight is 679.85 g/mol. Telaprevir has the following structural formula:

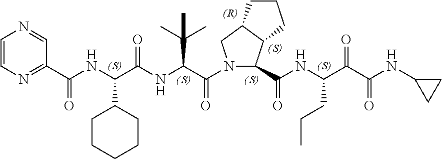

Telaprevir drug substance is a white to off-white powder with a solubility in water of 0.0047 mg/mL. Telaprevir interconverts to an R-diastereomer, VRT-127394, which is the major metabolite in plasma and is approximately 30-fold less potent than telaprevir. See Incivek™ Tablet Patient Prescribing Information.

The synthesis of boceprevir and telaprevir is known in the art and disclosed, for example, in U.S. Pat. Nos. 7,012,066 and 7,772,178 (boceprevir), as well as U.S. Pat. No. 7,820,671 (telaprevir), which are incorporated herein by reference.

Boceprevir and telaprevir can be administered by any of the routes and dosage forms commonly known in the art and enumerated above in the context of the description of ribavirin and its administration.

In some non-limiting embodiments of the present invention, the boceprevir dosage form is a hard gelatin capsules for oral administration containing 200 mg of boceprevir and the following inactive ingredients: sodium lauryl sulfate, microcrystalline cellulose, lactose monohydrate, croscarmellose sodium, pre-gelatinized starch, and magnesium stearate. The red capsule cap consists of gelatin, titanium dioxide, D&C Yellow #10, FD&C Blue #1, and FD&C Red #40. The yellow capsule body contains gelatin, titanium dioxide, D&C Yellow #10, FD&C Red #40, and FD&C Yellow #6. The capsule is printed with red and yellow ink. The red ink contains shellac and red iron oxide, while the yellow ink consists of shellac, titanium dioxide, povidone and D&C Yellow #10 Aluminum Lake. See Victrelis™ Capsule Patient Prescribing Information.

In some non-limiting embodiments of the present invention, the telaprevir dosage form is capsule-shaped, film-coated tablet for oral administration containing 375 mg of telaprevir and the following inactive ingredients: colloidal silicon dioxide, croscarmellose sodium, D&C Red No. 40, dibasic calcium phosphate (anhydrous), FD&C Blue No. 2, hypromellose acetate succinate, microcrystalline cellulose, polyethylene glycol, polyvinyl alcohol, sodium lauryl sulfate, sodium stearyl fumarate, talc, and titanium dioxide. See Incivek™ Tablet Patient Prescribing Information.

Boceprevir and telaprevir are approved for the treatment of chronic hepatitis C genotype 1 infection, in combination with pegylated interferon alfa and ribavirin, at doses of 750 mg (provided in divided doses of 375 mg) and 800 mg (provided in divided doses of 200 mg), respectively, administered three times a day. The scope of the present invention covers these daily doses, but is not limited to them. In some embodiments of the present invention, the daily dose of boceprevir or telaprevir is 100 mg to 200 mg. In other embodiments of the present invention, the daily dose of boceprevir or telaprevir is 200 mg to 400 mg, 400 mg to 600 mg, 600 mg to 800 mg, 800 mg to 1100 mg, 1100 mg to 1400 mg, 1400 mg to 1500 mg, 1500 mg to 1800 mg, 1800 mg to 2100 mg, 2100 mg to 2400 mg, 2400 mg to 2700 mg, 2700 mg to 3000 mg, 3000 mg to 3300 mg, 3300 mg to 3600 mg, or 3600 mg to 4000 mg.

Similarly, the scope of the present invention covers the approved boceprevir and telaprevir dosage regimens, but is not limited to them. In some embodiments of the present invention, boceprevir or telaprevir are administered once a week. In other embodiments of the present invention, boceprevir or telaprevir are administered twice a week. In other embodiments of the present invention, boceprevir or telaprevir are administered three times a week. In other embodiments of the present invention, boceprevir or telaprevir are administered once a day. In other embodiments of the present invention, boceprevir or telaprevir are administered twice a day. In other embodiments of the present invention, boceprevir or telaprevir are administered three times per day

Similarly, the scope of the present invention covers the approved strengths of individual dosage units of boceprevir and telaprevir, but is not limited to them. In some embodiments of the present invention, boceprevir or telaprevir is administered in dosage units comprising 10 mg to 40 mg of the active pharmaceutical ingredient. In other embodiments of the present invention, boceprevir or telaprevir are administered in dosage units comprising 40 mg to 80 mg, 80 mg to 120 mg, 120 mg to 160 mg, 160 mg to 200 mg, 200 mg to 240 mg, 240 mg to 280 mg, 280 mg to 320 mg, 320 mg to 360 mg, 360 mg to 400 mg, 400 mg to 450 mg, 450 mg to 500 mg, 500 mg to 550 mg, 550 mg to 600 mg, 600 mg to 700 mg, 700 mg to 800 mg, 800 mg to 900 mg, 900 mg to 1000 mg, 1000 mg to 1100 mg, or 1100 mg to 1200 mg of the active pharmaceutical ingredient.

Non-limiting examples of suitable dosages, dosage regimens and strengths of individual dosage units of boceprevir and telaprevir within the scope of the present invention, include any dosages, dosage regimens and strengths of individual dosage units that provide a therapeutic benefit to the subject, as recognized by the person having ordinary skill in the art. The amount of direct-acting antivirals, including but not limited to boceprevir and telaprevir, that will be effective, i.e., provide a therapeutic benefit, in the treatment and/or management of hepatitis C can be determined by as set forth above in the context of the description of ribavirin.

Additional direct acting antivirals within the scope of this invention are described below. HCV protease inhibitors also include, without limitation, ITMN-191, SCH-900518, TMC-435, BI-201335, MK-7009, VX-500, VX-813, BMS790052, BMS650032, VBY376, R7227, VX-985, ABT-333, ACH-1625, GS-9256, GS-9451, MK-5172 and ABT-450.

HCV helicase inhibitors include, but are not limited to,

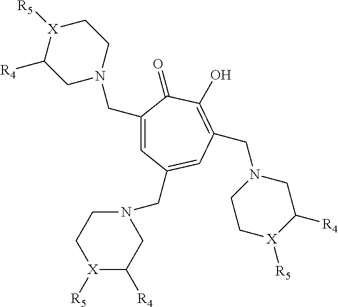

wherein X=N, R4=H and R5=CH3, X=CH, R4=H and R5=CH3, or X=CH, R4=CH3 and R5=H (see Gemma at al., 2010, Bioorg. Med. Chem. Lett. in press).

Another NS3 helicase inhibitor within the scope of the present invention has the following structure:

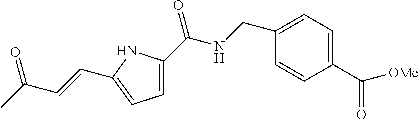 [SMILES: COC(=O)c1ccc(CNC(=O)c2ccc(/C=C/C(C)=O)[nH]2)cc1]

(see, Kandil et al., 2009, Bioorg. Med. Chem. Lett. 19(11), 2935-7).

Another NS3 inhibitor within the scope of the present invention has the following structure:

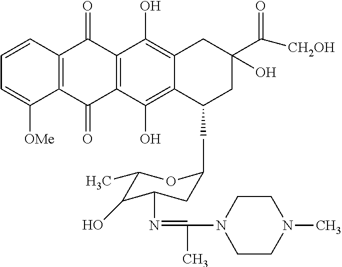 [SMILES: COc1cccc2c1C(=O)c1c(O)c3c(c(O)c1C2=O)CC(O)(C(=O)CO)C[C@@H]3CC1CC(/N=C(/C)N2CCN(C)CC2)C(O)C(C)O1]

### (see Krawczyk et al., 2009, Biol Chem. 390(4), 351-60).

Another NS3 helicase inhibitor within the scope of the present invention has the following structure:

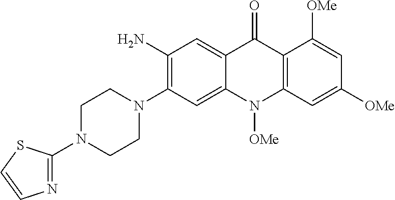 [SMILES: COc1cc(OC)c2c(=O)c3cc(N)c(N4CCN(c5nccs5)CC4)cc3n(OC)c2c1]

(see Manfroni et al., 2009, J. Med. Chem. 52(10), 3354-65).

Yet another NS3 helicase inhibitor within the scope of the present invention has the following structure:

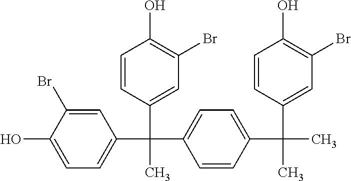 [SMILES: CC(C)(c1ccc(C(C)(c2ccc(O)c(Br)c2)c2ccc(O)c(Br)c2)cc1)c1ccc(O)c(Br)c1]

(see Chen et al., 2009, J. Med. Chem. 52, 2716-23).

Inhibitors of the HCV NS4B protein include, but are not limited to, clemizole, and other NS4B-RNA binding inhibitors, including but not limited to benzimidazole RBIs (B-RBIs) and indazole RBIs (I-RBIs) Inhibitors of HCV NS5A within the scope of the present invention include also, but are not limited to, BMS-790052, A-689, A-831, EDP239, GS5885, GSK805, PP1461, BMS-824393 and ABT-267.

Inhibitors of the HCV polymerase (NSSB) within the scope of the present invention include, but are not limited to, nucleoside analogs (e.g., valopicitabine, R1479, R1626, R7128), nucleotide analogs (e.g., IDX184, PSI-352938), and non-nucleoside analogs (e.g., filibuvir, HCV-796, VCH-759, VCH-916, ANA598, VCH-222 (VX-222), BI-207127, MK-3281, ABT-072, ABT-333, GS9190, BMS791325, INX-08189).

In some embodiments, the direct-acting antiviral within the scope of the present invention is the HCV NSSB polymerase inhibitor PSI-7851, which is a mixture of the two diastereomers PSI-7976 and PSI-7977. See Sofia et al., J. Med. Chem., 2010, 53:7202-7218; see also Murakami et al, J. Biol. Chem., 2010, 285:34337-34347. In other embodiments, the direct-acting antiviral within the scope of the present invention is PSI-7976 or PSI-7977. PSI-7851 has the structural formula depicted in the formula below:

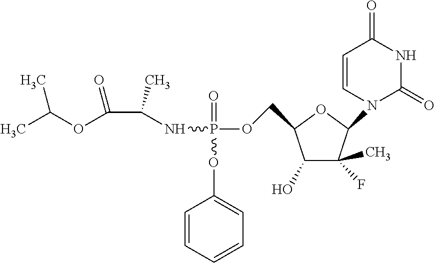

The molecular formula of PSI-7851 is C₂₂H₂₉FN₃O₉P and its molecular weight is 529.45 g/mol. Compound PSI-7976 has the structural formula depicted in the formula below:

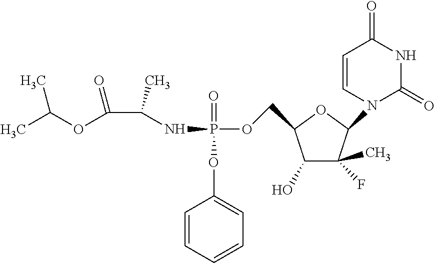 [SMILES: CC(C)OC(=O)[C@H](C)N[P@@](=O)(OC[C@H]1O[C@@H](n2ccc(=O)[nH]c2=O)[C@](C)(F)[C@@H]1O)Oc1ccccc1]

Compound PSI-7977 has the structural formula depicted in the formula below:

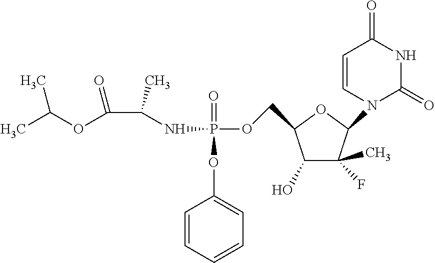 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

The CAS Registry Number of PSI-7977 is 1190307-88-0. Both racemic and non-racemic mixtures of compounds PSI-7976 and PSI-7977 are within the scope of the present invention.

Inhibitors of the HCV p7 protein within the scope of the present invention include, without limitation, BIT225 and HPH116.

IRES inhibitors within the scope of the present invention include, without limitation, Mifepristone, Hepazyme, ISIS14803, siRNAs/shRNAs.

HCV entry inhibitors within the scope of the present invention include, without limitation, HuMax HepC (an E2-antibody), JTK-652, PRO206, SP-30, and ITX5061.

An additional DAA within the scope of the present invention is Debio 025.

All of the direct acting antiviral compounds disclosed herein are available, commercially or otherwise, from sources known to those skilled in the art.

Non-limiting examples of suitable doses, dosage forms and dosing regimens, and routes of administration of the direct acting antiviral compounds disclosed herein within the scope of the present invention are the doses, dosage forms and dosing regimens, and the routes of administration set forth above in connection with the description of ribavirin.

The present invention provides a method of treating HCV infection, comprising administering ribavirin in combination with interferon. In some embodiments of the present invention, the interferon comprises interferon alfa-2α or interferon alfa-2β. In other embodiments of the present invention, the interferon comprises pegylated interferon alfa-2α or pegylated interferon alfa-2β. In even other embodiments of the present invention, the interferon comprises interferon alfacon-1 or pegylated interferon alfacon-1.

Interferon alfa-2α, interferon alfa-2β, pegylated interferon alfa-2α, pegylated interferon alfa-2β and interferon alfacon-1 have been approved for the treatment of chronic hepatitis C (CHC) virus infection in combination with ribavirin. The scope of the present invention is not restricted to the approved dosage regimens of interferon set forth below. In the context of the present invention, it is contemplated that interferon may be administered in any dose and dosage regimen, and by any route, that is suitable and beneficial for the subject. Non-limiting examples of suitable routes of administration within the scope of the present invention include intramuscular, subcutaneous, intraperitoneal or intravenous administration.

Non-limiting examples of suitable dosages, dosage regimens and strengths of individual dosage units of interferon within the scope of the present invention, include any dosages, dosage regimens and strengths of individual dosage units that provide a therapeutic benefit to the subject, as recognized by the person having ordinary skill in the art. The amount of interferon that will be effective, i.e., provide a therapeutic benefit, in the treatment and/or management of hepatitis C can be determined by as set forth above in the context of the description of ribavirin.

Non-limiting examples of suitable dosages of pegylated interferon alfa-2α for intramuscular, subcutaneous, intraperitoneal or intravenous administration within the scope of the present invention include interferon doses of about 180 μg per week for adult patients and 180 μg/1.73 m²×BSA per week, to a maximum dose of 180 μg, for pediatric patients. In some embodiments of the present invention, the pegylated interferon alfa-2a is administered to the subject once per week. In other embodiments of the present invention, the pegylated interferon alfa-2a is administered to the subject more than once per week. Where patients show an adverse reaction to interferon or suffer from certain conditions like depression, neutropenia, increased alanine transaminase (ALT) activity or decreased platelet counts, suitable dosages of pegylated interferon alfa-2a include doses of between about 90 and 180 μg of interferon per week. See Pegasys® Patient Prescribing Information.

Non-limiting examples of suitable dosages of nonpegylated interferon alfa-2a for intramuscular, subcutaneous, intraperitoneal or intravenous administration within the scope of the present invention include interferon doses of between about 16 μg per subject per week and about 233 μg per subject per week. In some embodiments of the present invention, about 11 μg of nonpegylated interferon alfa-2a is administered to a subject three times per week. In other embodiments of the present invention, about 22 μg of nonpegylated interferon alfa-2a is administered to a subject three times per week. In some embodiments of the present invention, nonpegylated interferon alfa-2a is administered three times per week. In other embodiments of the present invention, nonpegylated interferon alfa-2a is administered once per day. See Roferon® Patient Prescribing Information.

Non-limiting examples of suitable dosages of pegylated interferon alfa-2b for intramuscular, subcutaneous, intraperitoneal or intravenous administration within the scope of the present invention include interferon doses of between about 1.2 μg per kg bodyweight of the subject per week and about 1.6 μg per kg bodyweight of the subject per week. In some embodiments of the present invention, the interferon dose is about 1.5 μg per kg bodyweight of the subject per week. In some embodiments of the present invention, the pegylated interferon alfa-2b is administered to the subject once per week. In other embodiments of the present invention, the pegylated interferon alfa-2b is administered to the subject more than once per week. See Pegintron® Patient Prescribing Information.

Non-limiting examples of suitable dosages of nonpegylated interferon alfa-2b for intramuscular, subcutaneous, intraperitoneal or intravenous administration within the scope of the present invention include interferon doses of between about 9 million IU per subject per week and about 35 IU per subject per week. In some embodiments of the present invention, about 3 million IU of nonpegylated interferon alfa-2a are administered to a subject three times per week. In other embodiments of the present invention, about 10 million IU of nonpegylated interferon alfa-2a are administered to a subject three times per week. In other embodiments of the present invention, about 5 million IU of nonpegylated interferon alfa-2a are administered to a subject once a day. See Intron® Patient Prescribing Information.

Non-limiting examples of suitable dosages of interferon alfacon-1 for intramuscular, subcutaneous, intraperitoneal or intravenous administration within the scope of the present invention include interferon doses of between about 27 μg per subject per week and about 105 μg per subject per week. In some embodiments of the present invention, about 9 μg of interferon alfacon-1 is administered to a subject three times per week. In other embodiments of the present invention, about 15 μg of interferon alfacon-1 is administered to a subject three times per week. In other embodiments of the present invention, about 15 μg of interferon alfacon-1 is administered to a subject once a day. See Infergen® Patient Prescribing Information.

The scope of the present invention also includes the use of interferons other than pegylated or nonpegylated interferon alfa-2α, interferon alfa-2β or alfacon-1. Non-limiting examples of such interferons include other interferon alfa subtypes known in the art, as well as interferon beta, gamma and omega.

The term pegylated interferon as used herein means polyethylene glycol modified conjugates of interferon. Different types of pegylated and nonpegylated interferon alfa are commercially available from a variety of sources, as described above.

In some embodiments of the present invention, a dose of 1 to 5 μg interferon per kg bodyweight of the subject is administered per week. In other embodiments of the present invention, a dose of 5 to 10 μg interferon per kg bodyweight of the subject is administered per week. In other embodiments of the present invention, a dose of 10 to 15 μg interferon per kg bodyweight of the subject is administered per week. In other embodiments of the present invention, a dose of 15 to 20 μg interferon per kg bodyweight of the subject is administered per week. In other embodiments of the present invention, a dose of 20 to 25 μg interferon per kg bodyweight of the subject is administered per week. In other embodiments of the present invention, a dose of 25 to 30 μg interferon per kg bodyweight of the subject is administered per week. In other embodiments of the present invention, a dose of 30 to 35 μg interferon per kg bodyweight of the subject is administered per week. In other embodiments of the present invention, a dose of 35 to 40 μg interferon per kg bodyweight of the subject is administered per week.

In some embodiments of the present invention, interferon is administered once every two weeks. In other embodiments of the present invention, interferon is administered once per week. In other embodiments of the present invention, interferon is administered twice per week. In other embodiments of the present invention, interferon is administered three times per week. In other embodiments of the present invention, interferon is administered once per day. In other embodiments of the present invention, interferon is administered twice per day. In other embodiments of the present invention, interferon is administered three times per day.

Interferon may be administered in any suitable dosage form. Pharmaceutical compositions of pegylated or nonpegylated interferon suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or beraylalcohol), and surfactants (e.g. tween or polysorabates) in sterile water for injection. The pegylated or nonpegylated interferon may be stored as lyophilized powders under refrigeration at 2°-8° C. The reconstituted aqueous solutions are often stable when stored between 2°-8° C. and used within 24 hours of reconstitution. The reconstituted aqueous solutions may also be stored in prefilled, multi-dose syringes such as those useful for delivery of drugs such as insulin. Suitable syringes include systems comprising a prefilled vial attached to a pen-type syringe, as well as prefilled, pen-type syringes which allow easy self-injection by the user. Other syringe systems include a pen-type syringe comprising a glass cartridge containing a diluent and lyophilized pegylated interferon alfa powder in a separate compartment. See U.S. Pat. No. 6,849,254, which is incorporated herein by reference. See also the patents cited within U.S. Pat. No. 6,849,254.

The present invention also provides a method of treating HCV infection, comprising administering ribavirin, taribavirin, or derivatives thereof, in combination with both a direct-acting antiviral agent and interferon to a subject in need of such treatment. The doses, dosage forms and dosing regimens, and the routes of administration of ribavirin, taribavirin, or derivatives thereof, the direct-acting antiviral agent and the interferon in this embodiment of the present invention are in accordance with the doses, dosage forms and dosing regimens, and the routes of administration set forth above in connection with the descriptions of other embodiments of the present invention.

Bavituximab is a chimeric monoclonal antibody developed by Peregrine Pharmaceuticals, Inc. It binds to the cellular membrane component phosphatidylserine. Phosphatidylserine is usually located inside cells, but becomes exposed on the outside of the membranes of certain viruses and virally infected cells, creating a target for Bavituximab and antiviral therapy. Bavituximab, co-administered with ribavirin, is currently in phase II clinical trials. Methods of treating HCV infections by administering ribavirin in combination with Bavituximab, or other antibodies binding to phosphatidylserine, are also considered to be within the scope of the present invention. The determination of suitable doses, dosage forms and dosing regimens, and routes of administration of such antibodies is well within the routine skill of a person having ordinary skill in the art.

In non-limiting embodiments of the present invention, a sterile solution of Bavituximab, or another antibody binding to phosphatidylserine, is administered intravenously once per week at a dose of 0.3, 1, 3, or 6 mg/kg of bodyweight of the subject.

Non-limiting examples of suitable dosages, dosing regimens and strengths of individual dosage units of Bavituximab, within the scope of the present invention, include any dosages, dosing regimens and strengths of individual dosage units that provide a therapeutic benefit to the subject, as recognized by the person having ordinary skill in the art.

In some embodiments of the present invention, the enantiomer of ribavirin, also called L-ribavirin or levovirin, rather than ribavirin itself is administered to a subject to treat HCV infection in the subject. Levovirin has been reported to have immunomodulatory properties similar to those of ribavirin, but to be less toxic. See Watson J; Prospects for hepatitis C virus therapeutics: levovirin and viramidine as improved derivatives of ribavirin; Curr Opin Investig Drugs, 2002, 3(5):680-683; see also Lin C C et al.; Absorption, pharmacokinetics and excretion of levovirin in rats, dogs and Cynomolgus monkeys; Journal of Antimicrobial Chemotherapy, 2003, 51:93-99. The chemical name of levovirin is 1-β-L-ribofuranosyl-1H-1,2,4-triazole-3-carboxamide and levovirin has the structural formula depicted in the formula below:

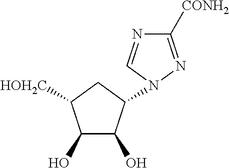 [SMILES: NC(=O)c1ncn([C@H]2C[C@@H](CO)[C@H](O)[C@@H]2O)n1]

The molecular formula of levovirin and its molecular weight are identical to the molecular formula and molecular weight of ribavirin. See U.S. Pat. No. 7,034,161.

The manufacture of levovirin is known in the art. The synthesis of levovirin is described, for example, in U.S. Patent Publication No. 20030092644, which is incorporated herein by reference in its entirety.

Non-limiting examples of suitable doses, dosage forms and dosing regimens, and routes of administration of levovirin within the scope of the present invention are the doses, dosage forms and dosing regimens, and the routes of administration set forth above in connection with the description of ribavirin. Similar to ribavirin, levovirin can be administered in combination with direct-acting antivirals, interferon, or both.

In some embodiments of the present invention, a derivative or prodrug of ribavirin, rather than ribavirin itself, is administered to a subject to treat HCV infection in the subject. A non-limiting example of a derivative or prodrug of ribavirin within the scope of the present invention is taribavirin. Other ribavirin derivatives include those disclosed in U.S. Pat. Nos. 7,638,496; 7,056,895; 6,930,093; 6,495,677; 6,455,508; 6,423,695; and 6,815,542, all of which are incorporated herein by reference in their entirety. Taribavirin (also known as viramidine, ribavirin amidine and ribamidine) is an amidine-derivative of ribavirin. Taribavirin has been developed by Valeant Pharmaceuticals International and it is currently in phase III clinical trials for the treatment of hepatitis C. The chemical name of taribavirin is 1-β-D-ribofuranosyl-1H-1,2,4-triazole-3-carboxamidine and taribavirin has the structural formula depicted in the formula below:

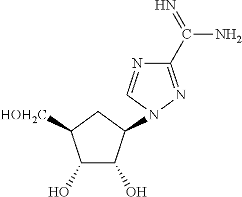 [SMILES: N=C(N)c1ncn([C@@H]2C[C@H](CO)[C@@H](O)[C@H]2O)n1]

The molecular formula of taribavirin is C₈H₁₃N₅O₄ and its molecular weight is 243.2 g/mol. A commonly used salt of taribavirin, and preferred embodiment of the present invention, is taribavirin hydrochloride, which has the molecular formula C₈H₁₄N₅O₄Cl₁.

The present invention provides a method of treating HCV infection, comprising administering taribavirin in combination with a direct-acting antiviral agent to a subject in need of such treatment, wherein the taribavirin is administered once daily at a dose of between 800 mg and 1600 mg.

The present invention further provides a method of treating HCV infection, comprising administering taribavirin in combination with interferon to a subject in need of such treatment, wherein between 800 mg and 1600 mg of taribavirin are administered once daily in a single unit.

In some embodiments of the present invention, treating HCV infections includes administering taribavirin in combination with both a direct-acting antiviral agent and interferon.

The present invention further provides a method of treating HCV infection, comprising administering taribavirin to a subject in need of such treatment, wherein taribavirin, or a pharmaceutically acceptable salt thereof, is administered once daily in a single unit comprising between 800 mg and 1600 mg of taribavirin.

The present invention further provides a pharmaceutical dosage unit comprising between 800 mg and 1600 mg of taribavirin.

The present invention further provides pharmaceutical dosage units, and the administration thereof, that comprise between about 1600 mg and about 4000 mg of taribavirin. In some embodiments of the present invention, the pharmaceutical dosage units comprises between about 1600 mg and about 2500 mg of taribavirin. In other embodiments of the present invention, the pharmaceutical dosage units comprises between about 2500 mg and about 4000 mg of taribavirin.

In some embodiments of the present invention, the taribavirin is administered at a dose of 20, 25 or 30 mg/kg/day.

The manufacture of taribavirin is known in the art. The synthesis of taribavirin is described, for example, in U.S. Pat. Nos. 6,495,677, 7,056,895 and 7,638,496, both of which are incorporated herein by reference in their entirety.

The scope of the present invention covers daily taribavirin doses of between 800 mg and 4000 mg, but is not limited to them. Non-limiting examples of suitable daily doses, within the scope of the present invention, include any daily doses that provide a therapeutic benefit to the subject, as recognized by the person having ordinary skill in the art. The amount of taribavirin that will be effective, i.e., provide a therapeutic benefit, in the treatment and/or management of hepatitis C can be determined by as set forth above in the context of the description of ribavirin.

Non-limiting examples of suitable doses, dosage forms and dosing regimens, and routes of administration of taribavirin within the scope of the present invention are the doses, dosage forms and dosing regimens, and the routes of administration set forth above in connection with the description of ribavirin. Similar to ribavirin, taribavirin can be administered in combination with direct-acting antivirals, interferon, or both. In some embodiments of the present invention, taribavirin is administered in an immediate release dosage unit. In other embodiments, taribavirin, is administered in an extended release dosage unit or a combination of an immediate release and an extended release dosage unit.

It is also contemplated within the scope of the present invention that HCV infections are treated by administering once daily dosage units that comprise both taribavirin and one or more direct-acting antivirals. Such dosage units can be manufactured and administered in ways generally known in the art. Non-limiting examples of suitable dosage forms and their routes of administration are the dosage forms and their routes of administration set forth above in connection with the description of taribavirin and the description of direct acting antivirals. In some embodiments of the present invention, the dosage unit that comprises both taribavirin and one or more direct-acting antivirals is a tablet or a capsule.

It is contemplated within the scope of the present invention that HCV infections are treated by administering once daily a dosage unit such as a tablet that comprises taribavirin and that is scored to facilitate dividing the dosage unit. It is also contemplated within the scope of the present invention that HCV infections are treated by administering a dosage unit such as a tablet that is segmented and that contains taribavirin in one and a direct-acting antiviral agent in another segment, wherein the dosage unit is scored to facilitate dividing it into the segment containing the taribavirin and the segment containing a direct-acting antiviral agent. The present invention contemplates that in the case where such segmented dosage units are used, the taribavirin-containing segments are administered once daily. In certain embodiments of the present invention, the scored dosage unit is an immediate release dosage unit. Techniques for scoring dosage units are generally known in the art and thus not described herein in further detail.

The present invention therefore contemplates and thus includes within its scope the use of ribavirin, taribavirin, levovirin, and any of the DAAs disclosed herein, including, but not limited to, boceprevir, telaprevir and PSI-7977, as well as any pharmaceutically acceptable salts, derivatives, isomers, stereoisomers, enantiomers, diastereomers, tautomers, racemates, non-racemic mixtures, solvates (including without limitation hydrates), acids, bases, esters, polymorphs and crystalline forms of any of the forgoing. As used herein and unless otherwise indicated, the term stereoisomer refers to compounds that possess identical constitution, but which differ in the arrangement of their atoms in space.

As used herein, the term pharmaceutically acceptable salt(s) includes pharmaceutically acceptable acid addition salts. Pharmaceutically acceptable acid addition salt are salts that retain the biological effectiveness of the free bases and that are not biologically or otherwise undesirable. The manufacture, selection and use of such acid addition salts is generally known by the person having ordinary skill in the art. Such pharmaceutically acceptable acid addition salts may be formed with a number of different acids, which include, but are not limited to, inorganic acids such as hydrochloric acid, hydrobromic acid, sulfuric acid, nitric acid, phosphoric acid, and the like, as well as organic acids such as acetic acid, trifluoroacetic acid, propionic acid, hexanoic acid, heptanoic acid, cyclopentanepropionic acid, glycolic acid, pyruvic acid, lactic acid, malic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, stearic acid, and the like. As used herein, the term pharmaceutically acceptable salt(s) also includes pharmaceutically acceptable base addition salts. The manufacture, selection and use of such salts is generally known by the person having ordinary skill in the art. Pharmaceutically acceptable base addition salts may be for example, but are not limited to, metallic salts made from aluminum, calcium, lithium, magnesium, potassium, sodium and zinc, or organic salts made from lysine, N,N′-dibenzylethylenediamine, chloroprocaine, choline, diethanolamine, ethylenediamine, meglumine (N-methylglucamine) and procaine. See, e.g., Remington's Pharmaceutical Sciences, 18th eds., Mack Publishing, Easton Pa. (1990) or Remington: The Science and Practice of Pharmacy, 19th eds., Mack Publishing, Easton Pa. (1995). In a preferred embodiment of the present invention, taribavirin is administered in the form of a hydrochloric acid salt.

The above-described dosage forms and administration schedules are provided for illustrative purposes only and should not be considered limiting. A person having ordinary skill in the art will readily understand that all suitable doses and administration schedules are within the scope of the present invention. It is to be understood and expected that variations in the principles of the invention disclosed herein may be made by one skilled in the art and it is intended that such modifications are to be included within the scope of the present invention.

The present invention provides once-a-day dosage forms and treatment regimens that are contemplated to improve the overall performance characteristics of ribavirin, taribavirin and other derivatives, and their pharmaceutically acceptable salts. In particular, it is envisioned that the present invention may increase patient compliance and may enhance early viral response (EVR) and sustained viral response (SVR) to treatment. It is also envisioned that the present invention may reduce gastrointestinal side effects, which may include for example nausea, vomiting, diarrhea, abdominal pain (dyspepsia) and early satiety. and that it may reduce the amount of concomitant medication required to treat these side effects.

Throughout this application, various publications are referenced. These publications are hereby incorporated into this application by reference in their entireties to more fully describe the state of the art to which this invention pertains.

The following examples further illustrate the invention, but should not be construed to limit the scope of the invention in any way.

### EXAMPLES

### Example 1

Film coated tablets containing 800, 1000 or 1200 mg of ribavirin were produced. The compositions of these tablets are set forth in Table 1 below:

TABLE 1

| | 800 mg | 1000 mg | 1200 mg |
|---|---|---|---|
| Ingredients | Tablet | Tablet | Tablet |
| Ribavirin, USP/PH.EUR | 800.0 | 1000.0 | 1200.0 |
| Microcrystalline Cellulose, | 168.7 | 210.9 | 253.1 |
| NF/PH.EUR/JP | | | |
| Lactose Monohydrate, NF/EP/JP | 60.0 | 74.9 | 89.9 |
| Croscarmellose Sodium, NF/EP | 40.0 | 50.1 | 60.1 |
| (Portion 1) | | | |
| Croscarmellose Sodium, NF/EP | 11.0 | 13.8 | 16.5 |
| (Portion 2) | | | |
| Povidone K 27-33, USP | 12.0 | 15.0 | 18.0 |
| Magnesium Stearate, NF/PH.EUR/JP | 8.3 | 10.3 | 12.4 |
| Purified Water USP/PH.EUR | Removed | Removed | Removed |
| | during | during | during |
| | processing | processing | processing |
| Core Tablet Weight (mg) | 1100.0 | 1375.0 | 1650.0 |
| Opadry II, Green Powder/85F110099 | 33.00 | — | — |
| Opadry II, Green Powder/85F110098 | — | 41.25 | — |
| Opadry II, Green Powder/85F110097 | — | — | 49.50 |
| Purified Water USP/PH.EUR | Removed | Removed | Removed |
| | during | during | during |
| | processing | processing | processing |
| Carnauba Wax, NF/PH.EUR | Trace | Trace | Trace |
| Film Coated Tablet Weight (mg) | 1133.00 | 1416.25 | 1699.50 |

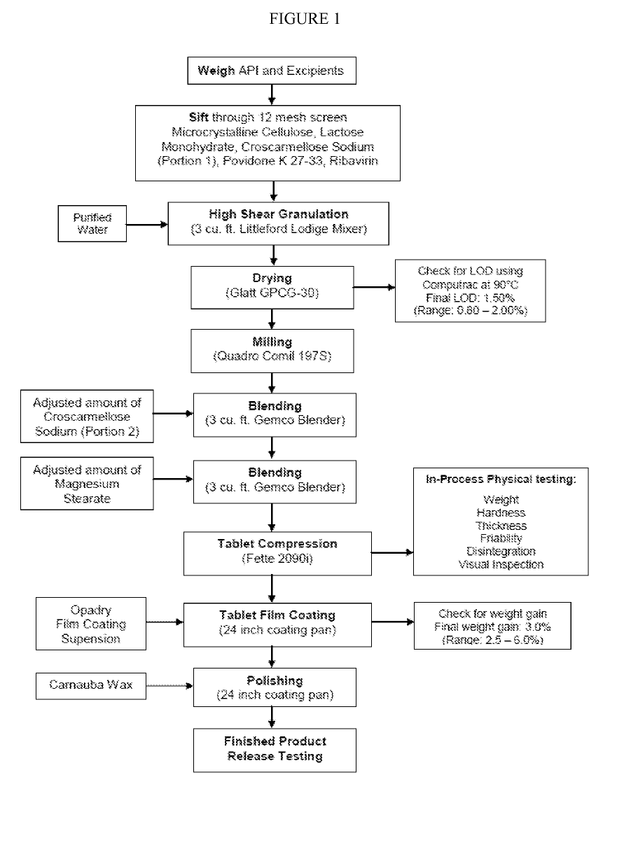

Overall, the 800, 1000 and 1200 mg ribavirin tablets were manufactured according to the process summarized in the flow chart depicted in FIG. 1. More specific information regarding individual steps of this process is provided below.

The granulation process was scaled down from commercial batch size of 350.0 kg to 24.5 kg. Pipe opening, without the spray tip nozzle, was used instead of gravity delivery nozzle to spray purified water into the granulator. Purified water was transferred to the granulator using a pressure pot. A pressure setting of 10 psi was used. At this 10 psi setting, the spray rate was approx. 5.2 kg/min. Granules were dried until the desired LOD was obtained. Final LOD values were as follows: 1.30% (800 mg ribavirin), 1.69% (1000 mg ribavirin) and 1.47% (1200 mg ribavirin). Dried granules were milled and then blended. No particular processing issues were observed during milling and blending.

The compression of the 800, 1000 and 1200 mg ribavirin tablets was performed according to the specifications provided in Table 2 below:

TABLE 2

| | 800 mg tablet | | 1000 mg tablet | | 1200 mg tablet | |
|---|---|---|---|---|---|---|
| Tablet Shape | Caplet | | | | | |
| Upper Punch | 0.8058 in × 0.3282 in | | 0.8682 in × 0.3537 in | | 0.9228 in × 0.3760 in | |
| | Embossed with | | Embossed with | | Embossed with | |
| | “800” | | “1000” | | “1200” | |
| Lower Punch | 0.8063 in × 0.3287 in | | 0.8687 in × 0.3542 in | | 0.9233 in × 0.3765 in | |
| | Embossed with “KDM” | | | | | |
| No of stations | 29 | | | | | |
| Fill Cam | 14 | mm | 16 | mm | 18 | mm |
| Press Speed | 40 | rpm | 35 | rpm | 25 | rpm |
| | 69,600 | tablets/hr | 60,900 | tablets/hr | 43,500 | tablets/hr |
| Theoretical Yield | 22,272 | tablets | 17,818 | tablets | 14,848 | tablets |

Often, the press speed was reduced to lessen tablet rejects during set-up and also due to low batch size. Hardness on the tablets was also increased, as the tablet weight increased. Some tablets showed sign of chipping on the edges, however there were no broken tablets. 800, 1000 and 1200 mg ribavirin tablets passed friability tests and weight, hardness and thickness values were holding up very well for these tablets. Set-up and in-process test results, obtained according to test procedures that are standard in the art, are shown in Tables 3-5 below.

TABLE 3
800 mg Ribavirin Tablets
Set-up Test results

| | |
|---|---|
| Weight (Total of 10 tabs) | % Friability |

| | | |
|---|---|---|
| 11.049 g | 11.036 g | 0.3% |

| | | |
|---|---|---|
| Hardness | Thickness (mm) | Disintegration |

| | | | |
|---|---|---|---|
| Average | 9.12 kP | 7.22 | 1 m 56 s |
| Min | 8.00 kP | 7.20 | 1 m 48 s |
| Max | 10.50 kP | 7.24 | 2 m 25 s |
| % srel | 7.46% | | |

In-Process Test Results

| | | | |
|---|---|---|---|
| | Beginning | Middle | End |
| Testing Frequency* | (3000 tabs) | (10,000 tabs) | (16,000 tabs) |

Weight

| | | | |
|---|---|---|---|
| Average | 1108.6 mg | 1124.0 mg | 1101.3 mg |
| Min | 1078.4 mg | 1090.3 mg | 1079.6 mg |
| Max | 1128.3 mg | 1139.6 mg | 1119.1 mg |
| % srel | 1.64% | 1.36% | 1.36% |

Hardness

| | | | |
|---|---|---|---|
| Average | 10.23 kP | 11.00 kP | 10.16 kP |
| Min | 8.80 kP | 8.50 kP | 7.60 kP |
| Max | 11.60 kP | 12.80 kP | 11.50 kP |
| % srel | 8.90% | 11.64% | 12.20% |

Thickness (mm)

| | | | |
|---|---|---|---|
| Average | 7.19 | 7.16 | 7.14 |
| Min | 7.15 | 7.13 | 7.12 |
| Max | 7.23 | 7.21 | 7.17 |

Friability (%)

| | | | |
|---|---|---|---|
| % Loss | Same as set-up | 0.1 | 0.1 |

*In-Process testing frequency changed to beginning, middle and end timepoint due to low batch size.

TABLE 4
1000 mg Ribavirin Tablets
Set-up Test results

| | |
|---|---|
| Weight (Total of 10 tabs) | % Friability |

| | | |
|---|---|---|
| 13.764 g | 13.841 g | 0.3% |

| | | |
|---|---|---|
| Hardness | Thickness (mm) | Disintegration |

| | | | |
|---|---|---|---|
| Average | 11.63 kP | 7.77 | 1 m 43 s |
| Min | 9.80 kP | 7.73 | 1 m 06 s |
| Max | 14.60 kP | 7.80 | 2 m 26 s |
| % srel | 14.79% | | |

In-Process Test Results

| | | | |
|---|---|---|---|
| | Beginning | Middle | End |
| Testing Frequency* | (1000 tabs) | (9,000 tabs) | (12,000 tabs) |

Weight**

| | | | |
|---|---|---|---|
| Average | 1391.5 mg | 1396.8 mg | 1.396 g |
| Min | 1348.0 mg | 1375.4 mg | 1.377 g |
| Max | 1423.3 mg | 1413.8 mg | 1.415 g |
| % srel | 2.20% | 0.95% | 0.99% |

Hardness

| | | | |
|---|---|---|---|
| Average | 12.16 kP | 12.37 kP | 12.10 kP |
| Min | 10.40 kP | 11.00 kP | 9.20 kP |
| Max | 14.30 kP | 13.10 kP | 15.00 kP |
| % srel | 11.43% | 5.82% | 13.47% |

Thickness (mm)

| | | | |
|---|---|---|---|
| Average | 7.76 | 7.75 | 7.74 |
| Min | 7.72 | 7.71 | 7.71 |
| Max | 7.80 | 7.79 | 7.78 |

Friability (%)

| | | | |
|---|---|---|---|
| % Loss | Same as set-up | 0.1 | 0.2 |

*In-Process testing frequency changed to beginning, middle and end timepoint due to low batch size.
**End timepoint sample was manually checked on balance for in-process weight test.

TABLE 5
1200 mg Ribavirin Tablets
Set-up Test results

| | |
|---|---|
| Weight (Total of 10 tabs) | % Friability |

| | | |
|---|---|---|
| 16.528 g | 16.660 g | 0.3% |

| | | |
|---|---|---|
| Hardness | Thickness (mm) | Disintegration |

| | | | |
|---|---|---|---|
| Average | 12.65 kP | 8.13 | 1 m 49 s |
| Min | 10.70 kP | 8.10 | 1 m 10 s |
| Max | 15.30 kP | 8.17 | 3 m 08 s |
| % srel | 11.86% | | |

In-Process Test Results

| | | | | |
|---|---|---|---|---|
| Testing | Beginning | | | End |
| Frequency* | (1,000 tabs) | 5,000 tabs | 10,000 tabs | (12,000 tabs) |

Weight

| | | | | |
|---|---|---|---|---|
| Average | 1660.2 mg | 1663.5 mg | 1663.4 mg | 1665.3 mg |
| Min | 1633.9 mg | 1644.2 mg | 1631.4 mg | 1633.0 mg |
| Max | 1698.6 mg | 1693.6 mg | 1689.6 mg | 1691.2 mg |
| % srel | 1.25% | 1.12% | 1.14% | 1.22% |

Hardness

| | | | | |
|---|---|---|---|---|
| Average | 14.00 kP | 12.95 kP | 13.26 kP | 12.98 KP |
| Min | 11.70 kP | 11.10 kP | 11.90 kP | 10.90 kP |
| Max | 15.20 kP | 15.40 kP | 14.50 kP | 14.60 kP |
| % srel | 8.43% | 9.34% | 7.32% | 9.86% |

Thickness (mm)

| | | | | |
|---|---|---|---|---|
| Average | 8.13 | 8.17 | 8.17 | 8.16 |
| Min | 8.09 | 8.11 | 8.12 | 8.15 |
| Max | 8.16 | 8.19 | 8.20 | 8.18 |

Friability (%)

| Testing | Beginning | Middle | End |
|---|---|---|---|
| Frequency** | (1,000 tabs) | (7,000 tabs) | (12,000 tabs) |
| % Loss | Same as set-up | 0.1 | 0.2 |

*In-Process testing frequency changed to every 5000 tablets. Sample was also pulled at the end of compression timepoint. Checkmaster was asked to manually sample the end timepoint sample at approx. 12,000 tablet production.
**Friability performed at beginning, middle and end timepoints.

The film coating of the 800 mg ribavirin tablets was done according to the following specifications. The compression yield was 18.2 kgs. It was determined that a 10 kg tablet load was optimal for a 24″ coating pan process. The initial set-points for the parameters used for the film coating were as follows: (1) air volume: 440 cfm; (2) exhaust temperature: 45° C.; (3) spray rate: approx. 29 g/min (at 31 mL/min pump setting); (4) distance from gun to bed: 5.75 inch; (4) atomization air pressure: 30 psi; (5) pan speed: 12 rpm.

Erosion of the tablet surface was sometimes observed when tablets were sampled from the coating pan for an in-process weight check. Initially, after about 30 minutes of spraying, the logo on the tablets started to fill. Logo filling usually occured due to spray drying. The first line of defense in such instances was to decrease the exhaust temperature. But the intent was to optimize the coating process, keeping exhaust temperature same as the commercial process. Hence, atomization air pressure was decreased to 25 psi and the spray rate was increased to approx. 30 g/min. Logo filling was still visible even after changing these parameters. So, after 40 minutes of spraying, the atomization air pressure was decreased further to 22 psi and the spray rate was increased to approx. 31 g/min. After 49 minutes of spraying, the gun to bed distance was decreased to 5.5 inches to reduce the occurrence of logo filling. Logos on the final coated tablets were discernible, even though logo filling was observed occasionally.

The film coating of the 1000 mg ribavirin tablets was done according to the following specifications. The compression yield was 16.4 kgs. Since logo filling issues were not resolved, it was decided to reduce the exhaust temperature and gun to bed distance for this batch. The initial set-points for the parameters used for the film coating were as follows: (1) air volume: 440 cfm; (2) exhaust temperature: (3) 40° C.; spray rate: approx. 30 g/min (at 28 mL/min pump setting); (4) distance from gun to bed: 5.0 inch; (5) atomization air pressure: 30 psi; (6) pan speed: 12 rpm.

Surface erosion was observed on the tablets after 3 minutes of spraying. Minor changes to process parameters were made to improve logo appearance. After 20 minutes, the spray rate was increased to approx. 32 g/min. After 30 minutes, the atom air pressure was decreased to 28 psi. After 50 minutes, the spray rate increased to 33 g/min. No logo filling issue occurred thereafter during processing.

The film coating of the 1200 mg ribavirin tablets was done according to the following specifications. The compression yield was 21.3 kgs. It was determined that a 10 kg tablet load was optimal for a 24″ coating pan process. Since surface erosion issues were still not resolved, it was decided to reduce the pan speed and/or increase spray rate for this batch. It also seemed that spray rate can be increased further, by increasing the exhaust temperature. The initial set-points for the parameters used for the film coating were as follows: (1) air volume: 440 cfm; exhaust temperature: 45° C.; (2) spray rate: approx. 40 g/min; (3) distance from gun to bed: 5.0 inch; (4) atomization air pressure: 33 psi; (5) pan speed: 10 rpm.

Surface erosion was observed on the tablets, when they were jogged before spraying commenced. Hence, it was asserted that surface erosion was mainly due to core tablet characteristic and not due to higher pan speed. The following changes were made during processing to improve logo appearance. After 8 minutes, the pan speed was increased to 12 rpm. After 10 minutes, the spray rate was increased to approx. 41 g/min. After 20 minutes, the atom air pressure was decreased to 32 psi. After 30 minutes, the spray rate was increased to approx. 42 g/min.

Another batch of 1200 mg ribavirin tablets was film coated using the following parameters: (1) air volume: 440 cfm; (2) exhaust temperature: 45° C.; (3) spray rate: approx. 42 g/min; (4) distance from gun to bed: 5.0 inch; (5) atomization air pressure: 33 psi; (6) pan speed: 10 rpm. After 2 minutes of spraying, the pan speed was increased to 12 rpm. All other process parameters were kept constant throughout the batch. Logos on the tablets looked clear with no sign of logo filling. No processing issues were encountered during this batch.

Release testing was performed according to the following testing methods: GVL-4088 (800 mg RBV), GVL-4089 (1000 mg RBV) and GVL-4090 (1200 mg RBV).

The manufactures RBV tablets had the following appearance: Un-scored capsule-shaped tablet with tan-like film coating. Debossed with logo “KDM” on one side and a logo “800,” “1000” or “1200” on the other.

The retention time of the major peak in the chromatogram of sample preparations obtained from the tablets prepared as described above corresponded to that of the major peak in the chromatogram of standard preparations. The moisture content of the tablets tested was between 1.4 and 1.6%. The amount of API in the tablets tested was between 99.1 and 100.3% of the label claim and the weight of the tablets tested was between 97.1 and 102.4% of the label claim. Also the dissolution profiles of the tablets met USP requirements. A small number of RBV-related substances were present in the manufacture tablets. The compound 1-β-D-Ribofuranosyl-1H-1,2,4-triazole-3-carboxylic acid (RTCOOH) was detected in the manufactured tablets in amounts of not more than 0.01%. The compound 1H-1,2,4-triazole-3-carboxylic acid (TCOOH) was was not detected in any manufactured tablets. And the compound 1H-1,2,4-triazole-3-carboxamide (TCONH2) was detected in the manufactured tablets in amounts of not more than 0.02%. Other unidentified contaminants were detected in the manufactured tablets in amounts of not more than 0.01%. In conclusion, all development batches of higher strength ribavirin tablets (i.e., 800, 1000 and 1200 mg) were manufactured without encountering major processing issues. Tablets met the required physical testing specifications and quality attributes. Tablets conformed to all the release testing specifications as well.

### Example 2

Film coated tablets containing 800, 1000 or 1200 mg of ribavirin were produced. The specifications of these tablets are set forth in Table 1 (800 mg), Table 2 (1000 mg) and Table 3 (1200 mg) below:

TABLE 1

| Test | Results | Specifications | Comments |
|---|---|---|---|
| Appearance | Conforms | Un-scored capsule-shaped | No Comments |
| | | tablet with tan-like film coating. | |
| | | Debossed with logo “KDM” | |
| | | on one side and logo “800” | |
| | | on the other. | |
| Identification (HPLC) | Conforms | The retention time of the | No Comments |
| | | major peak in the | |
| | | chromatogram of the Sample | |
| | | preparation corresponds to | |
| | | that of the major peak in the | |
| | | chromatogram of the Standard | |
| | | preparation. | |
| Moisture Assay | 1.6% | Not more than 4.0 percent | No Comments |
| | 99.6% l.c. | Not less than 90.0 percent and | No Comments |
| | | not more than 110.0 percent | |
| | | of label claim (equivalent to | |
| | | between 720 mg and 880 mg per | |
| | | tablet). | |
| Uniformity of | Conforms | Meets USP requirements. | No Comments |
| Dosage Units - | | | |
| Weight Variation | | | |
| Weight Variation | 101.5% | | No Comments |
| Result (Tablet 01) | | | |
| Weight Variation | 101.3% | | No Comments |
| Result (Tablet 02) | | | |
| Weight Variation | 99.4% | | No Comments |
| Result (Tablet 03) | | | |
| Weight Variation | 97.1% | | No Comments |
| Result (Tablet 04) | | | |
| Weight Variation | 101.4% | | No Comments |
| Result (Tablet 05) | | | |
| Weight Variation | 98.7% | | No Comments |
| Result (Tablet 06) | | | |
| Weight Variation | 101.6% | | No Comments |
| Result (Tablet 07) | | | |
| Weight Variation | 97.4% | | No Comments |
| Result (Tablet 08) | | | |
| Weight Variation | 99.2% | | No Comments |
| Result (Tablet 09) | | | |
| Weight Variation | 98.5% | | No Comments |
| Result (Tablet 10) | | | |
| Weight Variation | 99.6% | | No Comments |
| Result (Average) | | | |
| Weight Variation | 97.1% | | No Comments |
| Result (Minimum) | | | |
| Weight Variation | 101.6% | | No Comments |
| Result (Maximum) | | | |
| Weight Variation | 1.8% | | No Comments |
| Result (RSD) | | | |
| Weight Variation | 4.2 | Requirements are met if the | No Comments |
| Result (Acceptance | | acceptance value of the first | |
| Value) | | 10 dosage units is less than or | |
| | | equal to 15.0. | |
| Dissolution | Conforms | Meets USP requirements | No Comments |
| | | where Q = 80 percent label | |
| | | claim dissolved in 30 minutes. | |
| Dissolution Result | 100% | | No Comments |
| (Tablet 01) | | | |
| Dissolution Result | 100% | | No Comments |
| (Tablet 02) | | | |
| Dissolution Result | 98% | | No Comments |
| (Tablet 03) | | | |
| Dissolution Result | 102% | | No Comments |
| (Tablet 04 | | | |
| Dissolution Result | 99% | | No Comments |
| (Tablet 05 | | | |
| Dissolution Result | 101% | | No Comments |
| (Tablet 06 | | | |
| Dissolution Result | 100% | | No Comments |
| (Average) | | | |
| Dissolution Result | 98% | | No Comments |
| (Minimum) | | | |
| Dissolution Result | 102% | | No Comments |
| Result (Maximum) | | | |
| Dissolution Result | 1.4% | | No Comments |
| Result (RSD) | | | |
| Related Substances | 0.01% | Identified Individual: 1-β-D- | No Comments |
| | | Ribofuranosyl-1H-1,2,4- | |
| | | triazole-3-carboxylic acid | |
| | | (RTCOOH): Not more than | |
| | | 0.25 percent. | |
| Related Substances | 0.00% | Identified Individual: 1H- | Not Detected |
| | | 1,2,4-triazole-3-carboxylic | |
| | | acid (TCOOH): Not more | |
| | | than 0.25 percent. | |
| Related Substances | 0.01% | Identified Individual: 1H- | No Comments |
| | | 1,2,4-triazole-3-carboxamide | |
| | | (TCONH2): Not more than | |
| | | 0.25 percent. | |
| Related Substances | 0.01% | Unidentified Individual: Less | No Comments |
| | | than 0.10 percent. | |
| Related Substances | 0.0% | Total: Not more than 1.0 | 0.03694 |
| | | percent. | |

TABLE 2

| Test | Results | Specifications | Comments |
|---|---|---|---|
| Appearance | Conforms | Un-scored capsule-shaped | No Comments |
| | | tablet with light-green film coating. | |
| | | Debossed with logo “KDM” | |
| | | on one side and logo “1000” | |
| | | on the other. | |
| Identification (HPLC) | Conforms | The retention time of the | No Comments |
| | | major peak in the chromatogram | |
| | | of the Sample preparation | |
| | | corresponds to that of the major peak | |
| | | in the chromatogram of the Standard | |
| | | preparation. | |
| Moisture Assay | 1.5% | Not more than 4.0 percent | No Comments |
| | 99.1% l.c. | Not less than 90.0 percent and | No Comments |
| | | not more than 110.0 percent | |
| | | of label claim (equivalent to | |
| | | between 900 mg and 1100 mg per | |
| | | tablet). | |
| Uniformity of | Conforms | Meets USP requirements. | No Comments |
| Dosage Units - | | | |
| Weight Variation | | | |
| Weight Variation | 99.1% | | No Comments |
| Result (Tablet 01) | | | |
| Weight Variation | 99.1% | | No Comments |
| Result (Tablet 02) | | | |
| Weight Variation | 99.0% | | No Comments |
| Result (Tablet 03) | | | |
| Weight Variation | 101.8% | | No Comments |
| Result (Tablet 04) | | | |
| Weight Variation | 98.2% | | No Comments |
| Result (Tablet 05) | | | |
| Weight Variation | 98.1% | | No Comments |
| Result (Tablet 06) | | | |
| Weight Variation | 100.2% | | No Comments |
| Result (Tablet 07) | | | |
| Weight Variation | 100.2% | | No Comments |
| Result (Tablet 08) | | | |
| Weight Variation | 97.1% | | No Comments |
| Result (Tablet 09) | | | |
| Weight Variation | 98.2% | | No Comments |
| Result (Tablet 10) | | | |
| Weight Variation | 99.1% | | No Comments |
| Result (Average) | | | |
| Weight Variation | 97.1% | | No Comments |
| Result (Minimum) | | | |
| Weight Variation | 101.8% | | No Comments |
| Result (Maximum) | | | |
| Weight Variation | 1.4% | | No Comments |
| Result (RSD) | | | |
| Weight Variation | 3.2 | Requirements are met if the | No Comments |
| Result (Acceptance | | acceptance value of the first | |
| Value) | | 10 dosage units is less than or | |
| | | equal to 15.0. | |
| Dissolution | Conforms | Meets USP requirements | No Comments |
| | | where Q = 80 percent label | |
| | | claim dissolved in 30 minutes. | |
| Dissolution Result | 99% | | No Comments |
| (Tablet 01) | | | |
| Dissolution Result | 96% | | No Comments |
| (Tablet 02) | | | |
| Dissolution Result | 99% | | No Comments |
| (Tablet 03) | | | |
| Dissolution Result | 102% | | No Comments |
| (Tablet 04) | | | |
| Dissolution Result | 102% | | No Comments |
| (Tablet 05) | | | |
| Dissolution Result | 99% | | No Comments |
| (Tablet 06) | | | |
| Dissolution Result | 100% | | No Comments |
| (Average) | | | |
| Dissolution Result | 96% | | No Comments |
| (Minimum) | | | |
| Dissolution Result | 102% | | No Comments |
| (Maximum) | | | |
| Dissolution Result | 2.1% | | No Comments |
| (RSD) | | | |
| Related Substances | 0.01% | Identified Individual: 1-β-D- | No Comments |
| | | Ribofuranosyl-1H-1,2,4- | |
| | | triazole-3-carboxylic acid | |
| | | (RTCOOH): Not more than | |
| | | 0.25 percent. | |
| Related Substances | 0.00% | Identified Individual: 1H- | Not Detected |
| | | 1,2,4-triazole-3-carboxylic | |
| | | acid (TCOOH): Not more | |
| | | than 0.25 percent. | |
| Related Substances | 0.00% | Identified Individual: 1H- | 0.00743 |
| | | 1,2,4-triazole-3-carboxamide | |
| | | (TCONH2): Not more than | |
| | | 0.25 percent. | |
| Related Substances | 0.00% | Unidentified Individual: Less | 0.00495 |
| | | than 0.10 percent. | |
| Related Substances | 0.0% | Total: Not more than 1.0 | 0.01331 |
| | | percent. | |

TABLE 3

| Test | Results | Specifications | Comments |
|---|---|---|---|
| Appearance | Conforms | Un-scored capsule-shaped | No Comments |
| | | tablet with green film coating. | |
| | | Debossed with logo “KDM” | |
| | | on one side and logo “1200” | |
| | | on the other. | |
| Identification (HPLC) | Conforms | The retention time of the | No Comments |
| | | major peak in the | |
| | | chromatogram of the Sample | |
| | | preparation corresponds to | |
| | | that of the major peak in the | |
| | | chromatogram of the Standard | |
| | | preparation. | |
| Moisture Assay | 1.4% | Not more than 4.0 percent | No Comments |
| | 100.3% l.c. | Not less than 90.0 percent and | No Comments |
| | | not more than 110.0 percent | |
| | | of label claim (equivalent to | |
| | | between 1080 mg and 1320 mg | |
| | | per tablet). | |
| Uniformity of | Conforms | Meets USP requirements. | No Comments |
| Dosage Units - | | | |
| Weight Variation | | | |
| Weight Variation | 101.4% | | No Comments |
| Result (Tablet 01) | | | |
| Weight Variation | 100.4% | | No Comments |
| Result (Tablet 02) | | | |
| Weight Variation | 97.3% | | No Comments |
| Result (Tablet 03) | | | |
| Weight Variation | 102.4% | | No Comments |
| Result (Tablet 04) | | | |
| Weight Variation | 99.3% | | No Comments |
| Result (Tablet 05) | | | |
| Weight Variation | 98.7% | | No Comments |
| Result (Tablet 06) | | | |
| Weight Variation | 101.6% | | No Comments |
| Result (Tablet 07) | | | |
| Weight Variation | 101.2% | | No Comments |
| Result (Tablet 08) | | | |
| Weight Variation | 102.2% | | No Comments |
| Result (Tablet 09) | | | |
| Weight Variation | 98.5% | | No Comments |
| Result (Tablet 10) | | | |
| Weight Variation | 100.3% | | No Comments |
| Result (Average) | | | |
| Weight Variation | 97.3% | | No Comments |
| Result (Minimum) | | | |
| Weight Variation | 102.4% | | No Comments |
| Result (Maximum) | | | |
| Weight Variation | 1.8% | | No Comments |
| Result (RSD) | | | |
| Weight Variation | 4.2 | Requirements are met if the | No Comments |
| Result (Acceptance | | acceptance value of the first | |
| Value) | | 10 dosage units is less than or | |
| | | equal to 15.0. | |
| Dissolution | Conforms | Meets USP requirements | No Comments |
| | | where Q = 80 percent label | |
| | | claim dissolved in 30 minutes. | |
| Dissolution Result | 99% | | No Comments |
| (Tablet 01) | | | |
| Dissolution Result | 99% | | No Comments |
| (Tablet 02) | | | |
| Dissolution Result | 101% | | No Comments |
| (Tablet 03) | | | |
| Dissolution Result | 98% | | No Comments |
| (Tablet 04 | | | |
| Dissolution Result | 100% | | No Comments |
| (Tablet 05) | | | |
| Dissolution Result | 98% | | No Comments |
| (Tablet 06) | | | |
| Dissolution Result | 99% | | No Comments |
| (Average) | | | |
| Dissolution Result | 98% | | No Comments |
| (Minimum) | | | |
| Dissolution Result | 101% | | No Comments |
| Result (Maximum) | | | |
| Dissolution Result | 1.2% | | No Comments |
| Result (RSD) | | | |
| Related Substances | 0.01% | Identified Individual: 1-β-D- | No Comments |
| | | Ribofuranosyl-1H-1,2,4- | |
| | | triazole-3-carboxylic acid | |
| | | (RTCOOH): Not more than | |
| | | 0.25 percent. | |
| Related Substances | 0.00% | Identified Individual: 1H- | Not Detected |
| | | 1,2,4-triazole-3-carboxylic | |
| | | acid (TCOOH): Not more | |
| | | than 0.25 percent. | |
| Related Substances | 0.02% | Identified Individual: 1H- | No Comments |
| | | 1,2,4-triazole-3-carboxamide | |
| | | (TCONH2): Not more than | |
| | | 0.25 percent. | |
| Related Substances | 0.00% | Unidentified Individual: Less | 0.00424 |
| | | than 0.10 percent. | |
| Related Substances | 0.0% | Total: Not more than 1.0 | 0.02626 |
| | | percent. | |

What is claimed is:

1. A method of treating HCV infection, comprising administering ribavirin, or a pharmaceutically acceptable salt thereof, in combination with a direct-acting antiviral agent to a subject in need of such treatment, wherein the ribavirin, or a pharmaceutically acceptable salt thereof, is administered once daily at a dose of between 800 mg and 1400 mg.

2. The method of claim 1, wherein the dose of ribavirin, or a pharmaceutically acceptable salt thereof, is administered in one dosage unit.

3. The method of claim 1, wherein the dose of ribavirin, or a pharmaceutically acceptable salt thereof, is administered in two or three dosage units and wherein each dosage unit comprises 400, 500 or 600 mg of ribavirin, or a pharmaceutically acceptable salt thereof.

4. The method of claim 2, wherein the dosage unit of ribavirin, or a pharmaceutically acceptable salt thereof, is an immediate release dosage unit.

5. The method of claim 2, wherein the dosage unit of ribavirin, or a pharmaceutically acceptable salt thereof, is an extended release dosage unit or a combination of an immediate release dosage unit and an extended release dosage unit.

6. The method of claim 3, wherein at least one of the two or three dosage units of ribavirin, or a pharmaceutically acceptable salt thereof, is an immediate release dosage unit.

7. The method of claim 3, wherein at least one of the two or three dosage units of ribavirin, or a pharmaceutically acceptable salt thereof, is an extended release dosage unit or a combination of an immediate release dosage unit and an extended release dosage unit.

8. The method as in one of claims 1-7, wherein ribavirin, or a pharmaceutically acceptable salt thereof, is administered in the form of a tablet.

9. The method as in one of claims 1-7, wherein ribavirin, or a pharmaceutically acceptable salt thereof, is administered in the form of a capsule.

10. The method as in one of claims 1-7, wherein the direct-acting antiviral agent is telaprevir.

11. The method as in one of claims 1-7, wherein the direct-acting antiviral agent is boceprevir.

12. The method as in one of claims 1-7, wherein the direct-acting antiviral agent is a HCV protease inhibitor.

13. The method as in one of claims 1-7, wherein the direct-acting antiviral agent is a HCV helicase inhibitor.

14. The method as in one of claims 1-7, wherein the direct-acting antiviral agent is an inhibitor of HCV nonstructural protein 4B.

15. The method as in one of claims 1-7, wherein the direct-acting antiviral agent is an inhibitor of HCV nonstructural protein 5A.

16. The method as in one of claims 1-7, wherein the direct-acting antiviral agent is a HCV polymerase inhibitor.

17. The method as in one of claims 1-7, wherein the direct-acting antiviral agent is an inhibitor of HCV viral ion channel forming protein p7.

18. The method as in one of claims 1-7, wherein the subject is coinfected with HIV.

19. The method as in one of claims 1-7, wherein the subject is additionally administered interferon.

20. The method of claim 19, wherein the interferon is selected from the group consisting of interferon alfa-2α, interferon alfa-2β and alfacon-1, as well as pegylated interferon alfa-2α, interferon alfa-2β and alfacon-1.

21. A method of treating HCV infection, comprising administering ribavirin, or a pharmaceutically acceptable salt thereof, in combination with interferon to a subject in need of such treatment, wherein between 800 mg and 1400 mg of ribavirin, or a pharmaceutically acceptable salt thereof, are administered once daily in a single unit.

22. The method of claim 21, wherein the single unit is an immediate release dosage unit.

23. The method of claim 21, wherein the single unit is an extended release dosage unit or a combination of an immediate release dosage unit and an extended release dosage unit.

24. The method as in one of claims 21-23, wherein the interferon comprises interferon alfa-2α or interferon alfa-2β.

25. The method of claim 24, wherein the interferon is pegylated.

26. The method as in one of claims 21-23, wherein the interferon comprises interferon alfacon-1.

27. The method of claim 26, wherein the interferon is pegylated.

28. A method of treating HCV infection, comprising administering ribavirin, or a pharmaceutically acceptable salt thereof, to a subject in need of such treatment, wherein ribavirin, or a pharmaceutically acceptable salt thereof, is administered once daily in an immediate release single unit comprising between 800 mg and 1400 mg of ribavirin, or a pharmaceutically acceptable salt thereof.

29. The method of claim 28, wherein the immediate release single unit comprises 800 mg of ribavirin, or a pharmaceutically acceptable salt thereof.

30. The method of claim 28, wherein the immediate release single unit comprises 1000 mg of ribavirin, or a pharmaceutically acceptable salt thereof.

31. The method of claim 28, wherein the immediate release single unit comprises 1200 mg of ribavirin, or a pharmaceutically acceptable salt thereof.

32. The method as in one of claims 28-31, wherein the immediate release single unit is a tablet.

33. The method as in one of claims 28-31, wherein the immediate release single unit is a capsule.

34. A pharmaceutical dosage unit comprising between 800 mg and 1400 mg of ribavirin, or a pharmaceutically acceptable salt thereof, wherein the dosage unit is an immediate release dosage form.

35. The pharmaceutical dosage unit of claim 34, comprising 800 mg of ribavirin, or a pharmaceutically acceptable salt thereof.

36. The pharmaceutical dosage unit of claim 34, comprising 1000 mg of ribavirin, or a pharmaceutically acceptable salt thereof.

37. The pharmaceutical dosage unit of claim 34, comprising 1200 mg of ribavirin, or a pharmaceutically acceptable salt thereof.

38. The pharmaceutical dosage unit as in one of claims 34-37, wherein the dosage unit is a tablet.

39. The pharmaceutical dosage unit as in one of claims 34-37, wherein the dosage unit is a capsule.

40. A method of treating HCV infection, comprising administering taribavirin, or a pharmaceutically acceptable salt thereof, in combination with a direct-acting antiviral agent to a subject in need of such treatment, wherein the taribavirin, or a pharmaceutically acceptable salt thereof, is administered once daily at a dose of between 800 mg and 1600 mg.

41. The method of claim 34, wherein the dose of taribavirin, or a pharmaceutically acceptable salt thereof, is administered in one dosage unit.

42. The method of claim 34, wherein the dose of taribavirin, or a pharmaceutically acceptable salt thereof, is administered in more than one dosage units and wherein each dosage unit comprises 200 mg of taribavirin, or a pharmaceutically acceptable salt thereof.

43. The method of claim 41, wherein the dosage unit of taribavirin, or a pharmaceutically acceptable salt thereof, is an immediate release dosage unit.

44. The method of claim 41, wherein the dosage unit of taribavirin, or a pharmaceutically acceptable salt thereof, is an extended release dosage unit or a combination of an immediate release dosage unit and an extended release dosage unit.

45. The method of claim 42, wherein at least one of the dosage units of taribavirin, or a pharmaceutically acceptable salt thereof, is an immediate release dosage unit.

46. The method of claim 42, wherein at least one of the dosage units of taribavirin, or a pharmaceutically acceptable salt thereof, is an extended release dosage unit or a combination of an immediate release dosage unit and an extended release dosage unit.

47. The method as in one of claims 40-46, wherein taribavirin, or a pharmaceutically acceptable salt thereof, is administered in the form of a tablet.

48. The method as in one of claims 40-46, wherein taribavirin, or a pharmaceutically acceptable salt thereof, is administered in the form of a capsule.

49. The method as in one of claims 40-46, wherein the direct-acting antiviral agent is telaprevir.

50. The method as in one of claims 40-46, wherein the direct-acting antiviral agent is boceprevir.

51. The method as in one of claims 40-46, wherein the direct-acting antiviral agent is a HCV protease inhibitor.

52. The method as in one of claims 40-46, wherein the direct-acting antiviral agent is a HCV helicase inhibitor.

53. The method as in one of claims 40-46, wherein the direct-acting antiviral agent is an inhibitor of HCV nonstructural protein 4B.

54. The method as in one of claims 40-46, wherein the direct-acting antiviral agent is an inhibitor of HCV nonstructural protein 5A.

55. The method as in one of claims 40-46, wherein the direct-acting antiviral agent is a HCV polymerase inhibitor.

56. The method as in one of claims 40-46, wherein the direct-acting antiviral agent is an inhibitor of HCV viral ion channel forming protein p7.

57. The method as in one of claims 40-46, wherein the subject is coinfected with HIV.

58. The method as in one of claims 40-46, wherein the subject is additionally administered interferon.

59. The method of claim 58, wherein the interferon is selected from the group consisting of interferon alfa-2α, interferon alfa-2β and alfacon-1, as well as pegylated interferon alfa-2α, interferon alfa-2β and alfacon-1.

60. A method of treating HCV infection, comprising administering taribavirin, or a pharmaceutically acceptable salt thereof, in combination with interferon to a subject in need of such treatment, wherein between 800 mg and 1600 mg of taribavirin, or a pharmaceutically acceptable salt thereof, are administered once daily in a single unit.

61. The method of claim 60, wherein the single unit is an immediate release dosage unit.

62. The method of claim 60, wherein the single unit is an extended release dosage unit or a combination of an immediate release dosage unit and an extended release dosage unit.

63. The method as in one of claims 60-62, wherein the interferon comprises interferon alfa-2α or interferon alfa-2β.

64. The method of claim 63, wherein the interferon is pegylated.

65. The method as in one of claims 60-62, wherein the interferon comprises interferon alfacon-1.

66. The method of claim 65, wherein the interferon is pegylated.

67. A method of treating HCV infection, comprising administering taribavirin, or a pharmaceutically acceptable salt thereof, to a subject in need of such treatment, wherein taribavirin, or a pharmaceutically acceptable salt thereof, is administered once daily in a single unit comprising between 800 mg and 1600 mg of ribavirin, or a pharmaceutically acceptable salt thereof.

68. The method of claim 67, wherein the single unit comprises 800 mg of taribavirin, or a pharmaceutically acceptable salt thereof.

69. The method of claim 67, wherein the single unit comprises 1200 mg of taribavirin, or a pharmaceutically acceptable salt thereof.

70. The method of claim 67, wherein the single unit comprises 1600 mg of taribavirin, or a pharmaceutically acceptable salt thereof.

71. The method as in one of claims 67-70, wherein the single unit is a tablet.

72. The method as in one of claims 67-70, wherein the single unit is a capsule.

73. The method as in one of claims 67-70, wherein the single unit is an immediate release dosage unit.

74. The method as in one of claims 67-70, wherein the single unit is an extended release dosage unit.

75. A pharmaceutical dosage unit comprising between 800 mg and 1200 mg of taribavirin, or a pharmaceutically acceptable salt thereof.

76. The pharmaceutical dosage unit of claim 75, comprising 800 mg of taribavirin, or a pharmaceutically acceptable salt thereof.

77. The pharmaceutical dosage unit of claim 75, comprising 1200 mg of taribavirin, or a pharmaceutically acceptable salt thereof.

78. The pharmaceutical dosage unit of claim 75, comprising 1600 mg of taribavirin, or a pharmaceutically acceptable salt thereof.

79. The pharmaceutical dosage unit as in one of claims 75-78, wherein the dosage unit is a tablet.

80. The pharmaceutical dosage unit as in one of claims 75-78, wherein the dosage unit is a capsule.

81. The pharmaceutical dosage unit as in one of claims 75-78, wherein the dosage unit is an immediate release dosage unit.

82. The pharmaceutical dosage unit as in one of claims 75-78, wherein the dosage unit is an extended release dosage unit.
